SEQUENCE LISTING (16 sequences)

SEQ ID NO: 1 - protein, 9 aa, Artificial Sequence
  HERV epitope
FLQFKTWWI

SEQ ID NO: 2 - protein, 9 aa, Artificial sequence
  epitope 2
RLIPYDWEI

SEQ ID NO: 3 - protein, 9 aa, Artificial sequence
  epitope 3
KLIDCYTFL

SEQ ID NO: 4 - protein, 9 aa, Artificial sequence
  HERV epitope 4
YLSFIKILL

SEQ ID NO: 5 - protein, 9 aa, Artificial sequence
  HERV epitope 5
AMIPKDWPL

SEQ ID NO: 6 - protein, 9 aa, Artificial sequence
  HERV epitope 6
YAMSNLFSI

SEQ ID NO: 7 - protein, 9 aa, Artificial sequence
  HERV epitope 7
SMDDQLNQL

SEQ ID NO: 8 - protein, 29 aa, Artificial sequence
  peptide 8
KSKIKSKYASYLSFIKILLKRGGVKVSTK

SEQ ID NO: 9 - protein, 29 aa, Artificial sequence
  peptide 9
TLLDSIAHGHRLIPYDWEILAKSSLSPSQ

SEQ ID NO: 10 - protein, 29 aa, Artificial sequence
  peptide 10
LAKSSLSPSQFLQFKTWWIDGVQEQVRRN

SEQ ID NO: 11 - protein, 29 aa, Artificial sequence
  peptide 11
GPLQPGLPSPAMIPKDWPLLIIIDLKDCF

SEQ ID NO: 12 - protein, 29 aa, Artificial sequence
  peptide 12
KLIDCYTFLQAEVANAGLAIASDKIQTST

SEQ ID NO: 13 - protein, 29 aa, Artificial sequence
  peptide 13
WIRPTLGIPTYAMSNLFSILRGDSDLNSK

SEQ ID NO: 14 - protein, 29 aa, Artificial sequence
  peptide 14
RDVETALIKYSMDDQLNQLFNLLQQTVRK

SEQ ID NO: 15 - protein, 522 aa, Artificial sequence
  polypeptide (gag)
MGQTKSKIKSKYASYLSFIKILLKRGGVKVSTKNLIKLFQIIEQFCPWFPEQGTLDLKDWSQKETEGLHCEYVAEPVMAQSTQNVDYNQLQEVIYPETLKLEESKPRGTSPLPAGQVPVTLQPQKQVKENKTQPPVAYQYWPPAELQYRPPPESQYGYPGMPPAPQGRAPYPQPPTRRLNPTAPPSRQGSKLHEIAQEGEPPTVEARYKSFSIKKLKDMKEGVKQYGPNSPYMRTLLDSIAHGHRLIPYDWEILAKSSLSPSQFLQFKTWWIDGVQEQVRRNRAANPPVNIDADQLLGIGQNWSTISQQALMQNEAIEQVRAICLRAWEKIQDPSKEPYPDFVARLQDVAQKSIADEKARKVIVELMAYENANPECQSAIKPLKGKVPAGSDVISEYVKACDGIGGAMHKAMLMAQAITGVVLGGQVRTFGRKCYNCGQIGHLKKNCPVLNKQNITIQATTTGREPPDLCNEQRGQPQAPQQTGAFPIQPFVPQGFQGQQPPLSQVFQGISQLPQYNNCPPP

SEQ ID NO: 16 - protein, 831 aa, Artificial sequence
  polypeptide (pol)
NKSRKRRNRESLLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHCIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVLTKEQVRQAFINSGAWKIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHALTHVNAAGLKNKFDVTWKQAKDIVQHCTQCQVLHLPTQEAGVNPRGLCPNALWQMDVTHVPSFGRLSYVHVTVDTYSHFIWATCQSTSHVKKHLLSCFAVMGVPEKIKTDNGPGYCSKAFQKFLSQWKISHTTGIPYNSQGQAIVERTNRTLKTQLVKQKEGGDSKCTTPQMQLNLALYTLNFLNIYRNQTTTSAEQHLTGKKSPGENQLPVWIPTRHLKFYNEPIRDAKKSTSA

(19) United States
(12) Patent Application Publication
(10) Pub. No.: US 2021/0330774 A1
(43) Pub. Date: Oct. 28, 2021

(54) HERV-K-DERIVED ANTIGENS AS SHARED TUMOR ANTIGENS FOR ANTI-CANCER VACCINE

(71) Applicants: CENTRE LEON BERARD, Lyon (FR); Université Claude Bernard Lyon 1, VILLEURBANNE (FR); Centre national de la recherche scientifique, PARIS (FR); INSTITUT NATIONAL DE LA SANTE ET DE LA RECHERCHE MEDICALE (INSERM), PARIS (FR)

(72) Inventors: Stéphane DEPIL, LYON (FR); Laurie TONON, LYON (FR); Christophe CAUX, BRESSOLLES (FR); Paola BONAVENTURA, LYON (FR); Jenny VALLADEAU, MARENNES (FR)

(21) Appl. No.: 17/273,677

(22) PCT Filed: Sep. 6, 2019
(86) PCT No.: PCT/EP2019/073883

§ 371 Date: Mar. 4, 2021

(30) Foreign Application Priority Data
Sep. 6, 2018 (EP) 18306173.8

(51) Int. Cl. A61K 39/00; C07K 14/47; A61K 35/17; A61P 35/00

(52) U.S. Cl. CPC A61K 39/001184; A61P 35/00; A61K 35/17; C07K 14/4748

(57) ABSTRACT

A composition or vaccine comprising at least one peptide, or an expression vector that induces expression of said at least one peptide in vivo, the peptide consisting of, or comprising, shared HERV-K derived antigens, and a pharmaceutically acceptable vehicle or excipient. Composition comprising Cytotoxic T Lymphocytes (CTLs) of a patient treated with such a peptide, or comprising T-cell Receptor (TCR) engineered T cells recognizing such a peptide.

The present invention is related to the identification of epitopes or neoepitopes derived from HERV-K antigens that are shared in some tumor subtypes and that can be used in diagnosis, prognostic and immunomonitoring, as well as in antigenic compositions, immunogenic compositions, in anti-cancer vaccines or T cell-based immunotherapies. The invention thus relates to the fields of cancer and immunotherapy, and to the development of peptide-based antigenic immunogenic compositions including one or more, preferably several of these epitopes and useful in diagnosis, prognostic and immunomonitoring and in the treatment and prevention of cancer, and of immunogenic compositions and vaccines including one or more, preferably several of these epitopes for the treatment and prevention of cancer. As an alternative, the compositions of the invention comprise a vector or vectors performing or leading to expression of said peptides in vivo, for example the vectors may be DNA or RNA vectors, or bacterial or viral vectors.

### BACKGROUND OF THE INVENTION

Human endogenous retroviruses (HERVs) represent 8% of the human genome. They probably correspond to remnants of ancient germ line infections of exogenous retroviruses. Most HERV genes are non-functional due to DNA recombination, mutations, and deletions, but some produce functional proteins including group-specific antigen (Gag), polymerase (Pol) with reverse transcriptase, and the envelope (Env) surface unit. HERVs expression is repressed in normal cells by epigenetic mechanism.

HERVs have strong immunogenic properties linked to a <<viral mimicry>> and their expression is increased in some solid tumors due to demethylation. HERVs are believed to represent possible pathogenic agents in carcinogenesis, where they could act by insertional mutagenesis or involvement in chromosomal aberrations. Some HERV proteins, like HERV-K Rec and Np9, are also putative oncogenes.

The expression of HERVs in cancer has been associated with different effects on the immune system:

immunomodulation, through the immunosuppressive domain of the Env unit

activation of innate immunity by HERV dsRNA (triggering innate type I interferon signalling)

induction of adaptive immune responses against HERV antigens.

### SUMMARY OF THE INVENTION

Thus, in accordance with the present invention, there is provided isolated or purified peptides as well as compositions comprising at least one such peptide, or an expression vector that induces expression of said at least one such peptide in vivo, the peptide consisting of, or comprising, an epitope consisting of a sequence selected from the group consisting of the sequences FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7). The composition may further comprise an appropriate liquid, buffer or vehicle. In the compositions for use in the treatment of a human being, the composition further comprises a pharmaceutically acceptable vehicle, carrier or excipient.

There is in particular provided an immunogenic composition comprising at least one peptide, or an expression vector that induces expression of said at least one peptide in vivo, the peptide consisting of, or comprising, an epitope consisting of a sequence selected from the group consisting of the sequences FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO:6), SMDDQLNQL (SEQ ID NO: 7), and a pharmaceutically acceptable vehicle, carrier or excipient.

There is also provided a vaccine or anti-cancer vaccine comprising at least one peptide, or an expression vector that induces expression of said at least one peptide in vivo, the peptide consisting of, or comprising, an epitope consisting of a sequence selected from the group consisting of the sequences FLQFKTWWI (SEQ ID NO: 1), RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO:6), SMDDQLNQL (SEQ ID NO: 7), and a pharmaceutically acceptable vehicle, carrier or excipient.

The compositions according to the invention may in particular comprise: 2, 3, 4, 5, 6 or 7 peptides, or one or more expression vector(s) that induce(s) expression of said 2, 3, 4, 5, 6 or 7 peptides in vivo, the peptides having from 9 to 100 amino acid residues, each one comprising at least one, in particular one, of the epitopes of sequences SEQ ID NO: 1-7, and each peptide comprising at least one different epitope with respect to the others; or

at least one peptide, or an expression vector that induces expression of said at least one peptide in vivo, said peptide having from 9 to 100 amino acids residues and comprising 2, 3, 4, 5, 6 or 7 of the epitopes of sequences SEQ ID NO: 1-7.

In an embodiment, the compositions according to the invention may in particular comprise:

2, 3, 4, 5, 6 or 7 peptides, or one or more expression vector(s) that induce(s) expression of said 2, 3, 4, 5, 6 or 7 peptides in vivo, the peptides having from 9 to 100 amino acid residues, one comprising the epitope of sequence SEQ ID NO: 1 or 6, and at least one another comprising at least one of the other epitopes of sequences SEQ ID NO: 1-7, and each peptide comprising at least one different epitope with respect to the others; or

at least one peptide, or an expression vector that induces expression of said at least one peptide in vivo, said peptide having from 9 to 100 amino acids residues and comprising 2, 3, 4, 5, 6 or 7 of the epitopes of sequences SEQ ID NO: 1-7, including the epitope of sequence SEQ ID NO: 1 or 6, in particular both.

As it will be presented herein after, the compositions according to the invention may comprise the following embodiments (however other embodiments will also appear from the rest of the description):

the composition comprises 2, 3, 4, 5, 6, or the 7 peptides of sequences SEQ ID NO: 1 to 7; or it comprises one or more expression vectors inducing the in vivo expression of these peptides; in an embodiment, peptide of sequence SEQ ID NO: 1 or 6 or both these peptides is/are present or expressed;

the composition comprises a peptide which comprises 9 to 100 amino acid residues and at least one of said peptides of sequence SEQ ID NO: 1 to 7; the peptide may comprise 2, 3, 4, 5, 6, or 7 of the disclosed epitopes; in an embodiment, the composition may comprise the gag epitopes and/or the pol epitopes, as disclosed herein, or at least two or three of these epitopes of gag and/or pol; or it comprises one or more expression vectors inducing the in vivo expression of this or these peptides; in an embodiment, the peptide comprised or expressed includes the peptide of sequence SEQ ID NO: 1 or 6, or both these peptides;

the composition comprises 2, 3, 4, 5, 6, or 7 peptides having from 9 to 100 amino acid residues, each one comprising at least one, preferably one, of the epitopes of sequences SEQ ID NO: 1 to 7, and each peptide comprises at least one different epitope with respect to the others; or it comprises one or more expression vectors inducing the in vivo expression of these peptides; in an embodiment, peptide of sequence SEQ ID NO: 1 or 6, or both these peptides is/are present or expressed;

each contained or expressed peptide of 9 to 100 amino acid residues comprises one specific (different from the other peptides in the composition) epitope of SEQ ID NO: 1, 2, 3, 4, 5, 6 or 7;

the contained or expressed peptide(s) comprise(s) 9 to 50 amino acid residues of an HERV gag or pol including at least one of said peptides of sequence SEQ ID NO: 1 to 7;

the composition comprises 1, 2, 3, 4, 5, 6 or 7 peptides selected from the group consisting of the peptides of SEQ ID NO: 8 to 14; or it comprises one or more expression vectors inducing the in vivo expression of this peptide or these peptides; in an embodiment, peptide of sequence SEQ ID NO: 8 or 13, or both these peptides is/are present or expressed.

Epitopes of sequences SEQ ID NO: 4, 2, and 1 are from HERV-K gag. Epitopes of sequences SEQ ID NO: 5, 3, 6 and 7 are from HERV-K pol. These are MHC class I HLA-A2 epitopes. In an embodiment, the composition comprises or expresses 1, 2 or the 3 HERV-K gag epitopes. In an embodiment, the composition comprises or expresses 1, 2, 3 or the 4 HERV-K pol epitopes.

In the context of the composition, immunogenic composition or vaccine in accordance with the invention, the contained or expressed peptide may comprise 9 to 100, in particular 9 to 70, or 9 to 50, 40, 30, 25, 20, consecutive residues, preferably those residues are from HERV-K gag and/or pol, more preferably a native consensus HERV-K gag and/or pol sequence, including at least one of the above-described epitopes. The peptide may be less than 50 residues in length, such as 9, 10, 11, 12, 13, 14, 15, 16, 17, 18, 19, 20, 25, 30, 35, 40, or 45 residues in length.

The composition, immunogenic composition or vaccine (hereinafter designated as “the composition” unless indicated to the contrary) in accordance with the invention may comprise more than one of these peptides of HERV-K, especially from gag and/or pol, or an expression vector that induces expression of in vivo more than one of these peptides of HERV-K, especially from gag and/or pol, or several (more than one) expression vectors each inducing expression in vivo of a different peptide (of these peptides) of HERV-K, especially from gag and/or pol.

In an embodiment, the peptide (having more than 9 residues) is a native HERV-K fragment comprising the 9-mer epitope and adjacent amino acids at the N-terminal and/or C-terminal forming the peptide of a given length. In an embodiment, the peptide comprises more than two HERV-K epitopes (e.g. 2, 3, 4, 5, 6 or 7 of the disclosed epitopes).

In an embodiment, the peptide (having more than 9 residues) is a native HERV-K fragment comprising the 9-mer epitope and adjacent amino acids at the N-terminal and/or C-terminal, and additional foreign amino acids forming the peptide of a given length. In an embodiment, the peptide comprises more than two HERV-K epitopes (e.g. 2, 3, 4, 5, 6 or 7 of the disclosed epitopes).

In another embodiment, all or part of the peptide sequence of the peptides contained in the composition or expressed, is foreign to HERV-K. In this embodiment, a peptide may easily comprise more than two HERV-K epitopes, e.g. 2, 3, 4, 5, 6 or 7 of the disclosed epitopes. The length of the peptide is suited to comprise the number of epitopes, and possible additional amino acids. The peptide may thus be of 9 or 10 to 69 peptide length, or more.

Preferably, the composition comprises or the vector induces expression of, 2, 3, 4, 5, 6 or 7 different peptides, each peptide comprising or consisting of a different epitope consisting of a sequence selected from the group consisting of the sequences FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7).

In an embodiment, the composition comprises or the vector induces expression of, at least one peptide comprising or consisting of at least two different epitopes consisting of a sequence selected from the group consisting of the sequences FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7). In an embodiment, the peptide comprises 2, 3, 4, 5, 6 or 7 of said epitopes; or the expression vector induces expression of a peptide comprising 2, 3, 4, 5, 6 or 7 of said epitopes.

Several solutions exist to have the different epitopes represented in the composition or in the expression products in vivo in the same patient. The composition may comprise or the vector induces expression of one or more peptides comprising one or more of the different epitopes of said group, so that 2, 3, 4, 5, 6 or 7 of said epitopes FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7), are present in the composition or expressed.

When speaking about an expression vector inducing expression of more than one peptide in accordance with the invention, it is possible to have the composition comprising a vector inducing expression of several peptides (wherein the peptide may comprise one epitope of the group, or more than 1, e.g 2, 3, 4, 5, 6, 7 of said epitopes), or at least two expression vectors, wherein the several vectors each induces expression of at least one peptide. In an embodiment, the composition comprises one single vector or several vectors, and the vector(s) induces expression of one or more peptides and 2, 3, 4, 5, 6 or 7 of said epitopes.

Examples of isolated or purified 29-mer peptides comprising epitopes and other gag or pol amino acid residues according to the invention are:

(SEQ ID NO: 8)
KSKIKSKYASYLSFIKILLKRGGVKVSTK,
(SEQ ID NO: 9)
TLLDSIAHGHRLIPYDWEILAKSSLSPSQ,
(SEQ ID NO: 10)
LAKSSLSPSQFLQFKTWWIDGVQEQVRRN,
(SEQ ID NO: 11)
GPLQPGLPSPAMIPKDWPLLIIIDLKDCF,
(SEQ ID NO: 12)
KLIDCYTFLQAEVANAGLAIASDKIQTST,
(SEQ ID NO: 13)
WIRPTLGIPTYAMSNLFSILRGDSDLNSK,
and
(SEQ ID NO: 14)
RDVETALIKYSMDDQLNQLFNLLQQTVRK.

Each one of these isolated or purified 29-mer peptides or fragments or 28 to 10 amino acid residues, comprising a 9-mer epitope, and under isolated or purified form, is an object of the invention. The peptide may be less than 29 residues in length, such as 9, 10, 11, 12, 13, 14, 15, 16, 17, 18, 19, 20, 21, 22, 23, 24, 25, 26, 27, 28 residues of those sequences SEQ ID NO: 8-14, including the 9-mer epitope. Interestingly, the amino acid sequences added to the epitopes may comprise, as it is the case in the peptides of SEQ ID NO: 8-14, other potential CD4 and/or CD8 T epitopes. The present invention provides for peptides comprising an epitope as disclosed herein and “further amino acids” at the C-terminal and/or N-terminal end. These further amino acids may be gag or pol sequences as disclosed herein with the sequences 1-16. However, the invention encompasses variation of amino acids at the level of these “further amino acids” within these gag/pol sequences. Thus, the invention encompasses those sequences, including sequences 1-16, wherein the further amino acid sequences have an identity percentage with those gag/pol sequences of at least 80, 85, 90, 91, 92, 93, 94, 95, 96, 97, 98 or 99%.

In an embodiment, the compositions of the invention comprise or express the peptide of SEQ ID NO: 8, or the peptide of SEQ ID NO: 13, or both peptides of SEQ ID NO: 8 and 13, and 1, 2, 3, 4, 5 of the other peptides of SEQ ID NO: 8-14.

In an embodiment, the composition further comprises an adjuvant.

Peptide RLIPYDWEI (SEQ ID NO: 2) under isolated or purified form is an object of the invention.

Peptide KLIDCYTFL (SEQ ID NO: 3) under isolated or purified form is an object of the invention.

Peptide YLSFIKILL (SEQ ID NO: 4) under isolated or purified form is an object of the invention.

Peptide AMIPKDWPL (SEQ ID NO: 5) under isolated or purified form is an object of the invention.

Peptide YAMSNLFSI (SEQ ID NO: 6) under isolated or purified form is an object of the invention.

Peptide SMDDQLNQL (SEQ ID NO: 7) under isolated or purified form is an object of the invention.

Peptides of 10 to 100 amino acids comprising at least one of said peptides of SEQ ID NO: 1-7, under isolated or purified form are an object of the invention.

A peptide comprising 2, 3, 4, 5, 6 or 7 of FLQFKTWWI (SEQ ID NO: 1), RLIPYDWEI (SEQ ID NO: 2), KLIDCYTFL (SEQ ID NO: 3), YLSFIKILL (SEQ ID NO: 4), AMIPKDWPL (SEQ ID NO: 5), YAMSNLFSI (SEQ ID NO: 6), and SMDDQLNQL (SEQ ID NO: 7) epitopes, under isolated or purified form, is an object of the invention. Said peptide preferably comprise FLQFKTWWI (SEQ ID NO: 1) and/or YAMSNLFSI (SEQ ID NO: 6).

In an embodiment, this peptide may comprise 9 to 100, in particular 9 to 70, or 9 to 50, 40, 30, 25, 20, or even 10 to 30, 12-25, preferably 14-18, e.g. 14, 15, 16, 17, or 18, consecutive residues, preferably of HERV-K gag and/or pol, more preferably a native consensus HERV-K gag and/or pol sequence, including at least one of the above-described epitopes, with the amino acids in addition to the corresponding epitope of sequence SEQ ID NO: 1-7, being the native amino acids as present in the HERV-K gag or pol as disclosed herein, and extending in 5′, 3′ or 5′ and 3′ of said epitope. The peptide may be less than 50 residues in length, such as 9, 10, 11, 12, 13, 14, 15, 16, 17, 18, 19, 20, 25, 30, 35, 40, or 45 residues in length, typically 14, 15, 16, 17, or 18. Another object of the invention is an expression vector comprising a nucleic acid encoding an amino acid sequence selected from the group consisting of FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7), and at least two of them (especially 2, 3, 4, 5, 6 or 7 of them), and elements necessary for leaving to the in vivo expression of the nucleic acid (polynucleotide) in a patient.

An example of HERVK-gag polypeptide comprising the 3 gag epitopes:

(SEQ ID NO: 15)
MGQTKSKIKSKYASYLSFIKILLKRGGVKVSTKNLIKLFQIIEQFCPWFP
EQGTLDLKDWSQKETEGLHCEYVAEPVMAQSTQNVDYNQLQEVIYPETLK
LEESKPRGTSPLPAGQVPVTLQPQKQVKENKTQPPVAYQYWPPAELQYRP
PPESQYGYPGMPPAPQGRAPYPQPPTRRLNPTAPPSRQGSKLHEIAQEGE
PPTVEARYKSFSIKKLKDMKEGVKQYGPNSPYMRTLLDSIAHGHRLIPYD
WEILAKSSLSPSQFLQFKTWWIDGVQEQVRRNRAANPPVNIDADQLLGIG
QNWSTISQQALMQNEAIEQVRAICLRAWEKIQDPSKEPYPDFVARLQDVA
QKSIADEKARKVIVELMAYENANPECQSAIKPLKGKVPAGSDVISEYVKA
CDGIGGAMHKAMLMAQAITGVVLGGQVRTFGRKCYNCGQIGHLKKNCPVL
NKQNITIQATTTGREPPDLCNEQRGQPQAPQQTGAFPIQPFVPQGFQGQQ
PPLSQVFQGISQLPQYNNCPPP

An example of HERVK-pol polypeptide comprising the 4 pol epitopes:

(SEQ ID NO: 16)
NKSRKRRNRESLLGAATVEPPKPIPLTWKTEKPVWVNQWPLPKQKLEALH
LLANEQLEKGHIEPSFSPWNSPVFVIQKKSGKWRMLTDLRAVNAVIQPMG
PLQPGLPSPAMIPKDWPLIIIDLKDCFFTIPLAEQDCEKFAFTIPAINNK
EPATRFQWKVLPQGMLNSPTICQTFVGRALQPVREKFSDCYIIHCIDDIL
CAAETKDKLIDCYTFLQAEVANAGLAIASDKIQTSTPFHYLGMQIENRKI
KPQKIEIRKDTLKTLNDFQKLLGDINWIRPTLGIPTYAMSNLFSILRGDS
DLNSKRMLTPEATKEIKLVEEKIQSAQINRIDPLAPLQLLIFATAHSPTG
IIIQNTDLVEWSFLPHSTVKTFTLYLDQIATLIGQTRLRIIKLCGNDPDK
IVVLTKEQVRQAFINSGAWKIGLANFVGIIDNHYPKTKIFQFLKLTTWIL
PKITRREPLENALTVFTDGSSNGKAAYTGPKERVIKTPYQSAQRAELVAV
ITVLQDFDQPINIISDSAYVVQATRDVETALIKYSMDDQLNQLFNLLQQT
VRKRNFPFYITHIRAHTNLPGPLTKANEQADLLVSSALIKAQELHALTHV
NAAGLKNKFDVTWKQAKDIVQHCTQCQVLHLPTQEAGVNPRGLCPNALWQ
MDVTHVPSFGRLSYVHVTVDTYSHFIWATCQSTSHVKKHLLSCFAVMGVP
EKIKTDNGPGYCSKAFQKFLSQWKISHTTGIPYNSQGQAIVERTNRTLKT
QLVKQKEGGDSKCTTPQMQLNLALYTLNFLNIYRNQTTTSAEQHLTGKKS
PGENQLPVWIPTRHLKFYNEPIRDAKKSTSA

Isolated or purified polypeptides of SEQ ID NO: 15 and 16 are objects of the invention, as are compositions, immunogenic compositions and anti-cancer vaccines as defined herein and comprising or expressing the polypeptide (s) of SEQ ID NO: 15 and/or SEQ ID NO: 16.

In an embodiment, the expression vector comprising a nucleic acid encoding a 9 to 100, in particular 9 to 70, or 9 to 50, 40, 30, 25, 20, amino acid peptide (the encoded peptide may be less than 50 residues in length, such as 9, 10, 11, 12, 13, 14, 15, 16, 17, 18, 19, 20, 25, 30, 35, 40, or 45 residues in length), comprising an epitope selected from the group consisting of FLQFKTWWI (SEQ ID NO: 1) RLIPYDWEI (SEQ ID NO: 2) KLIDCYTFL (SEQ ID NO: 3) YLSFIKILL (SEQ ID NO: 4), AMIPKDWPL (SEQ ID NO: 5), YAMSNLFSI (SEQ ID NO: 6), SMDDQLNQL (SEQ ID NO: 7), and the elements necessary for the in vivo expression of the nucleic acid (polynucleotide) in a patient. As mentioned above, the vector may comprise a nucleic acid sequence such that the vector induces expression of 2, 3, 4, 5, 6 or 7 epitope-containing peptides, or induces expression of a peptide comprising 2, 3, 4, 5, 6 or 7 of said epitopes. Also, as mentioned above, the vector may comprise a nucleic acid encoding a peptide comprising one or several of these epitopes, and amino acid residues in addition to the concerned epitope, wherein the additional residues may be from pol or gag, or being foreign to gag or pol.

The expression construct or vector may a non-viral expression construct, such as a bacterial expression construct, a DNA or RNA expression construct, or a viral expression construct. The expression construct may be located in an antigen-presenting cell. The construct may lead to integration of the expression construct into the genome of the cell.

The present invention also concerns these compositions for use in treating cancer. Cancers concerned by this use may be selected particularly (however without limitation) from those cancers: breast cancer, including triple negative breast cancer, ovarian cancer, melanoma, sarcoma, teratocarcinoma, bladder cancer, lung cancer (non small cell lung carcinoma and small cell lung carcinoma), head and neck cancer, colo-rectal cancer, glioblastoma, and leukemias, etc, for example breast cancer, including triple negative breast cancer, ovarian cancer, melanoma, sarcoma, teratocarcinoma, bladder cancer and leukemias.

The use may aim at activating T-cell responses, B-cell response or both in patients, for example in breast cancer patients.

The present invention also concerns the use of one 9-mer epitope or several of the 9-mer epitopes as disclosed herein, or of one peptide or several peptides as disclosed herein, or of one expression vector or several expression vectors as disclosed herein, or of a composition as disclosed herein, for the manufacture of an immunogenic composition or vaccine to treat a cancer as disclosed herein.

Another object of the invention is a method for treating cancer, comprising administering to a patient in need thereof a therapeutically effective amount of an immunogenic composition or vaccine as disclosed herein. As explained above, the composition may comprise the peptide or peptides, or one or more expression vectors or constructs. The method may comprise administering the vaccine more than once. The therapeutically effective daily amount of peptide (total amount of peptide or peptides according to the invention) administered may be in the range of 0.01 mg to 10 mg, 0.025 mg to 5.0 mg, or in the range of 0.025 mg to 1.0 mg.

Another object of the invention is a method for treating cancer in a patient comprising (a) contacting Cytotoxic T Lymphocytes (CTLs) of a patient in need of cancer treatment with a composition or immunogenic composition according to the invention; and (b) administering a therapeutically effective amount of the CTLs of step (a) to the patient. The method may further comprise expanding said CTL's by ex vivo or in vivo methods prior to administration. Contacting may comprise providing an antigen-presenting cell loaded with the peptide(s) of the invention or expressing said peptide(s) or polypeptide(s) from an expression construct. The therapeutically effective amount of CTL cells required to provide therapeutic benefit may be from about 0.1×10⁴ to about 5×10⁹ cells per kilogram weight of the subject. The method may comprise performing step (b) more than once.

The invention also concerns a method of preparing CTLs comprising contacting Cytotoxic T Lymphocytes (CTLs) of a patient in need of cancer treatment with a composition or immunogenic composition according to the invention, and possibly expanding said CTL's ex vivo. Contacting may comprise providing an antigen-presenting cell loaded with the peptide(s) of the invention or expressing said peptide(s) or polypeptide(s) from an expression construct.

A composition comprising such CTLs in a pharmaceutical vehicle, prepared as disclosed supra, is also an object of the invention.

Another object of the invention is T-cell Receptor (TCR) engineered T cells recognizing epitope peptides from the invention and compositions comprising such T cells in a pharmaceutical vehicle. The process of preparing these T cells is known from the skilled person. It may be the following (and the process is also an object of the invention): (i) TCR α and β chains are isolated from T cells recognizing epitope peptides from the invention and inserted into a vector (lentivirus or retrovirus for instance); (ii) T cells isolated from the peripheral blood of a patient or a donor are modified with such a vector (lentivirus or retrovirus for instance) to encode the desired TCRαβ sequences; (iii) these modified T cells are then expanded in vitro to obtain sufficient numbers for treatment and administered into the patient. Of note, TCR sequences can be modified for optimization of TCR affinity. The method of use of these T cells, e.g. treatment of cancer, is another object of the invention, and comprises administering to a subject in need thereof an efficient amount of those T cells. The therapeutically effective amount of T cells required to provide therapeutic benefit may be from about 0.1×10⁴ to about 5×10⁹, cells per kilogram weight of the subject.

In an embodiment, the cancer is triple negative breast cancer (TNBC), other breast cancers, ovarian cancer, melanoma, sarcoma, teratocarcinoma, bladder cancer, lung cancer (non small cell lung carcinoma and small cell lung carcinoma), head and neck cancer, colo-rectal cancer, glioblastoma and leukemias for example breast cancer, including triple negative breast cancer, ovarian cancer, melanoma, sarcoma, teratocarcinoma, bladder cancer and leukemias.

The antigens made or comprising the epitopes as disclosed herein also may be used to generate anti-HERV-K antibodies and to detect the presence of anti-HERV-K antibodies in HERV-K+ cancer patients.

The epitopes and the composition comprising at least one antigenic peptide according to the invention may be used in diagnosis, prognostic or immunomonitoring methods. In particular, the present invention also concerns a method for the immunomonitoring of immune response in a patient. Induction of an antitumor adaptive response after immunotherapy (vaccine using the epitopes or a composition of the invention or any other immunotherapy inducing adaptive antitumor T cell response) will be evaluated by measurement of specific T cell responses against the HERV epitopes of the invention. Measurement can be performed for instance by using multimers containing the epitopes described in the invention, directly or after ex vivo stimulation with peptides described in the invention. Measurement of T cell response can be also performed after ex vivo stimulation with peptides of the invention by using FACS analysis, ELISA, ELISPOT or other method to detect specific T cell activation.

In an embodiment, the biological sample is blood, blood derivative containing circulating cells or lymphocytes from the tumor. Preferably, the method comprises determining that some lymphocytes in the blood can specifically recognize and/or be specifically reactivated against the peptides of interest upon in vitro stimulation.

One of ordinary skill would know various assays to determine whether an immune response against a tumor-associated peptide was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

### DETAILED DESCRIPTION

The inventors localized HERVs sequences on the human genome and developed RNAseq analysis of HERVs. Using RNAseq data of 84 breast cancer from a public database, of which 42 triple negative breast cancer (TNBC) and 42 from ER+ subtype, they compared this expression with RNAseq from normal breast tissue samples, of which 51 from peritumoral area and 5 from mammal reduction samples. 19 HERVs were specifically overexpressed in TNBC, the majority of them belonging to HERV-K family.

Multiple component analysis showed that HERVs can be used to characterize the triple negative subtype. HERVs expression is associated with higher OCT4 (POU5F1) and lower TRIM28 levels in TNBC, two factors that regulate positively and negatively, respectively, the transcription of HERVs. A link with EMT signature was also observed, which may be associated with stemness features in TNBC. Interestingly, HERVs expression significantly correlated with T cells and cytotoxic lymphocytes transcriptomic signature, which may be explained by a type I interferon (IFN) response and the presence of antigen presenting cells signature. The effector T cell signature was counterbalanced by an immunomodulatory signature (including negative immune checkpoints and IDO1/2) and suppressor cells (including regulatory T cells and MDSCs).

The polymorphism of HERVs is often considered as a major obstacle to characterize T cell response against a specific HERV antigen or to use them in a strategy of cancer vaccination. Based on the specific expression of a limited number of HERVs characterizing TNBC, hypothesis was made that it may be possible to identify common regions inside the Gag and Pol proteins shared between different HERVs expressed in TNBC and then to determine T cell epitopes present in these domains. Common regions in Gag and in Pol from several HERV-K overexpressed in TNBC and containing intact ORF for each protein were effectively found. Interestingly, these shared domains contain several regions enriched in potential strong epitope binders for the most frequent MHC class I and II alleles, using different epitope prediction tools (including NetMHC I and II).

9-mer peptides corresponding to predicted HLA-A2 epitopes were synthetized and used for an in vitro protocol, consisting in the stimulation of peripheral blood mononuclear cells (PBMCs) to induce a specific response against the peptides of interest. The presence of specific CD8+ T cell was evaluated by multimer staining and the functional response (IFN gamma production and degranulation) was further evaluated against T2 cells pulsed with the cognate peptide, showing a specific activation of CD8+ T cells against HERV peptides made in accordance with the invention. Furthermore, the cytotoxicity of HERV-specific CD8+ T cells against a HERV-expressing tumor cell line was demonstrated using CD8+ T cells specific of the peptide SEQ ID NO 1, confirming the functional antitumor properties of the T cells generated by these peptides.

Considering the enhanced HERV expression in tumor cells and the results obtained these conclusions can be made:

HERVs are preferentially expressed in tumors and 19 HERVs subtypes characterize triple negative breast cancer (TNBC), most of them belonging to HERV-K family.

Common sequences containing T cell epitopes can be found between these 19 HERVs subtypes.

Seven 9-mer peptides were identified as strong HLA-A2 binders and able to elicit a specific CD8+ T cell response, with a specific cytotoxic response against T2 cells pulsed with the cognate peptide or against a HERV-expressing tumor cell line: FLQFKTWWI (SEQ ID NO: 1), RLIPYDWEI (SEQ ID NO: 2), KLIDCYTFL (SEQ ID NO: 3), YLSFIKILL (SEQ ID NO: 4), AMIPKDWPL (SEQ ID NO: 5), YAMSNLFSI (SEQ ID NO: 6), SMDDQLNQL (SEQ ID NO: 7).

HERVs products represent thus shared tumor antigens capable of inducing functional T cell responses. HERV-derived tumor antigens can be used for the development of cancer vaccines and to monitor adaptive immune responses.

### Definitions

The phrases “isolated”, “purified”, or “biologically pure” refer to material which is substantially or essentially free from components which normally accompany the material as it is found in its native state. Thus, isolated peptides in accordance with the invention preferably do not contain materials normally associated with the peptides in their in situ environment.

“Major histocompatibility complex” or “MHC” is a cluster of genes that plays a role in control of the cellular interactions responsible for physiologic immune responses. In humans, the MHC complex is also known as the HLA complex. For a detailed description of the MHC and HLA complexes

“Human leukocyte antigen” or “HLA” is a human class I or class II major histocompatibility complex (MHC) protein.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectable with a pharmaceutically acceptable usual vehicle or excipient, or carrier, either as liquid solutions or suspensions; solid forms suitable for solution in, or suspension in, liquid prior to injection can also be prepared.

As used herein, “vehicle, excipient, carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

“Immunogenic peptide” means that the peptide, once presented to the immune system in a patient, may induce an humoral and/or cellular immune response, and this response is immunogenic, but is not necessarily protective. This applies to an “immunogenic composition”.

An “immunogenic response” refers to a CTL and/or an HTL response to an antigen derived from an infectious agent or a tumor antigen. The immune response may also include an antibody response which has been facilitated by the stimulation of helper T cells.

In particular, the immunogenic composition may induce in vivo activation of CD8+ T cells against the HERV peptides present in the composition and/or against HERV peptides or polypeptides comprising similar epitopes that expressed in tumor cells.

“Vaccine composition” or “vaccinal peptide” means that once administered to a patient, respectively presented to the immune system in a patient, the composition or the peptide may induce an humoral and/or cellular immune response, and this immune response is protective.

A “protective immune response” refers to a CTL and/or an HTL response to an antigen derived from an infectious agent or a tumor antigen, which prevents or at least partially arrests disease symptoms or progression. The immune response may also include an antibody response which has been facilitated by the stimulation of helper T cells.

“Immunogenicity” means that the peptide or epitope, once present in the patient, especially in a patient's blood, tissue or organ, is able to induce a humoral and/or cell-mediated immune response.

By the composition or the vector “induces expression”, it is meant that it comprises an expression vector or expression vectors comprising a nucleic acid, DNA or RNA, coding for the peptide(s). The vector may be especially a RNA vector, a DNA vector or plasmid, a viral vector or a bacterial vector. There can be integration of an expression cassette into the host cell genome or there can be no integration, depending on the nature of the vector and as this is well known to the skilled person. The expression vector or the expression cassette may further comprise elements necessary for the in vivo expression of the nucleic acid (polynucleotide) in a patient. In minimum manner, this consists of an initiation codon (ATG), a stop codon and a promoter, as well as a polyadenylation sequence for certain vectors such as the plasmids and viral vectors other than poxviruses. The ATG is placed at 5′ of the reading frame and a stop codon is placed at 3′. As it is well-known, other elements making it possible to control the expression could be present, such as enhancer sequences, stabilizing sequences and signal sequences permitting the secretion of the peptide.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. Synthetic peptides will generally be about up 35 residues long, which is the approximate upper length limit of automated peptide synthesis machines, such as those available from Applied Biosystems (Foster City, Calif.). Longer peptides also may be prepared, e.g., by recombinant means.

A “peptide epitope” or “epitope” is a peptide which comprises an allele-specific motif or supermotif such that the peptide will bind an HLA molecule and induce a CTL and/or HTL response. Thus, immunogenic or vaccinal peptides of the invention, comprising at least one “peptide epitope” are capable of binding to an appropriate HLA molecule and thereafter inducing a cytotoxic T cell response, or a helper T cell response, to the antigen from which the immunogenic or vaccinal peptide is derived.

It is contemplated that peptides of the present invention may further employ amino acid sequence variants such as substitutional, insertional or deletion variants. Deletion variants lack one or more residues of the native protein. Insertional mutants typically involve the addition of material at a non-terminal point in the polypeptide. Substitutions are changes to an existing amino acid. These sequence variants may generate truncations, point mutations, and frameshift mutations. As is known to one skilled in the art, synthetic peptides can be generated by these mutations.

It also will be understood that amino acids sequence variants may include additional residues, such as additional N- or C-terminal amino acids, and yet still be essentially as set forth in one of the sequences disclosed herein, so long as the sequence meets the criteria set forth above, including the maintenance of biological activity.

The following is a discussion based upon changing the amino acids of a protein, such as a peptide or protein of the invention, to create a mutated, truncated, or modified protein. For example, certain amino acids may be substituted for other amino acids in the tumor-associated peptide or protein, resulting in a greater CTL immune response Since it is the interactive capacity and nature of a protein that defines that protein's biological functional activity, certain amino acid substitutions can be made in a protein sequence, and in its underlying nucleic acid coding sequence, thereby producing a mutated, truncated or modified protein.

In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Other Composition Components

In other embodiments of the invention, the composition may comprise an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bound to the peptide. Other immunopotentiating compounds are also contemplated for use with the compositions of the invention such as polysaccharides, including chitosan. Multiple (more than one) epitopes or peptides may be crosslinked to one another (e.g., polymerized).

The use of small peptides for immunization or vaccination, may also typically require conjugation of the peptide to a carrier peptide, polypeptide or protein conferring immunogenicity or strongest immunogenicity to the end product or the target peptide or epitope. Thus in an embodiment of the invention, each selected peptide among FLQFKTWWI (SEQ ID NO: 1), RLIPYDWEI (SEQ ID NO: 2), KLIDCYTFL (SEQ ID NO: 3), YLSFIKILL (SEQ ID NO: 4), AMIPKDWPL (SEQ ID NO: 5), YAMSNLFSI (SEQ ID NO: 6) and SMDDQLNQL (SEQ ID NO: 7), is conjugated or linked through peptide link to a peptide, polypeptide or protein (additional amino acid residues) that confers immunogenicity or strongest immunogenicity to the conjugated product or peptide of the invention.

In an embodiment, the peptide or epitope FLQFKTWWI (SEQ ID NO: 1), RLIPYDWEI (SEQ ID NO: 2), KLIDCYTFL (SEQ ID NO: 3), YLSFIKILL (SEQ ID NO: 4), AMIPKDWPL (SEQ ID NO: 5), YAMSNLFSI (SEQ ID NO: 6) and/or SMDDQLNQL (SEQ ID NO: 7) is present in a longer peptide or polypeptide, especially an HERV-K peptide or polypeptide, and preferably it is the natural longer peptide or polypeptide comprising the epitope in the HERV-K from which the epitope originates. Longer sequences are thus presented by way of example, with the sequences SEQ ID NO: 8-14. In a variant, several epitopes are part of the same longer peptide. It is also encompassed in the invention to conjugate those longer peptides to a peptide, polypeptide or protein that confers immunogenicity or strongest immunogenicity to the conjugated end product.

In the immunogenic composition or vaccine according to the invention, the peptides contained therein, or expressed by the vector(s), are immunogenic or able to induce a protective immune response.

If the composition or the peptide or epitope is used for diagnosis or assay purpose, such as immunomonitoring, then the peptide or epitope may be antigenic. Thus, in an embodiment of the composition, the peptide or epitope FLQFKTWWI (SEQ ID NO: 1), RLIPYDWEI (SEQ ID NO: 2), KLIDCYTFL (SEQ ID NO: 3), YLSFIKILL (SEQ ID NO: 4),

AMIPKDWPL (SEQ ID NO: 5), YAMSNLFSI (SEQ ID NO: 6) and/or SMDDQLNQL (SEQ ID NO: 7), or the peptide or epitope comprising such an epitope, is antigenic. The antigenic peptide or epitope may be in an unconjugated form (it consists of the epitope sequence SEQ ID NO: 1-7) or may be conjugated to a peptide or polypeptide moiety, as disclosed herein.

One of ordinary skill would know various assays to determine whether an immune response against a tumor-associated peptide was generated. The phrase “immune response” includes both cellular and humoral immune responses. Various B lymphocyte and T lymphocyte assays are well known, such as ELISAs, cytotoxic T lymphocyte (CTL) assays, such as chromium release assays, proliferation assays using peripheral blood lymphocytes (PBL), tetramer assays, and cytokine production assays. See Benjamini et al. (1991), hereby incorporated by reference.

Adjuvants

As also well known in the art, the immunogenicity of a particular immunogen composition can be enhanced by the use of non-specific stimulators of the immune response, known as adjuvants. Some adjuvants affect the way in which antigens are presented. For example, the immune response is increased when protein antigens are precipitated by alum. Emulsification of antigens also prolongs the duration of antigen presentation. Suitable molecule adjuvants include all acceptable immunostimulatory compounds, such as cytokines, toxins or synthetic compositions.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant. Other adjuvants that may also be used include IL-1, IL-2, IL-4, IL-7, IL-12, interferon, GM-CSF, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used.

In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol®) used as an about 0.25% solution. Adjuvant effect may also be made my aggregation of the antigen in the vaccine by heat treatment with temperatures ranging between about 70° to about 101° C. for a 30 second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA®) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. MDP stimulates macrophages but also appears to stimulate B cells directly.

In certain embodiments, hemocyanins and hemoerythrins may also be used in the invention. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described. Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated. U.S. Pat. No. 4,950,645 describes a lipophilic disaccharide-tripeptide derivative of muramyl dipeptide which is described for use in artificial liposomes formed from phosphatidyl choline and phosphatidyl glycerol.

BCG (bacillus Calmette-Guerin, an attenuated strain of Mycobacterium) and BCG-cell wall skeleton (CWS) may also be used as adjuvants, with or without trehalose dimycolate. Trehalose dimycolate may be used itself. BCG is an important clinical tool because of its immunostimulatory properties

Amphipathic and surface active agents, e.g., saponin and derivatives such as QS21 (Cambridge Biotech), form yet another group of adjuvants for use with the immunogens of the present invention. Nonionic block copolymer surfactants may also be employed. Oligonucleotides are another useful group of adjuvants. Quil A and lentinen are other adjuvants that may be used in certain embodiments of the present invention.

Another group of adjuvants are the detoxified endotoxins, such as the refined detoxified endotoxin of U.S. Pat. No. 4,866,034.

Those of skill in the art will know the different kinds of adjuvants that can be conjugated to cellular vaccines in accordance with this invention and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the invention.

Adjuvants may be encoded by a nucleic acid (e.g., DNA or RNA). It is contemplated that such adjuvants may be also be encoded in a nucleic acid (e.g., an expression vector) encoding the antigen, or in a separate vector or other construct. Nucleic acids encoding the adjuvants can be delivered directly, such as for example with lipids or liposomes. An example of such adjuvant is poly-ICLC.

Expression Vectors

The peptides according to the invention may be produced in vivo in body's patient.

An immunogenic composition or vaccine may contain RNA or DNA encoding one or more of the peptides as described above, such that the peptide is generated in situ. The RNA or the DNA may be present within any of a variety of delivery systems known to those of ordinary skill in the art, including nucleic acid expression systems (nude DNA or plasmid, RNA vector), bacterial or viral expression systems. Appropriate nucleic acid expression systems contain the necessary RNA or DNA sequences for expression in the patient (such as a suitable promoter and terminating signal). Bacterial delivery systems involve the administration of a bacterium (such as Bacillus-Calmette-Guerrin) that induces expression of an immunogenic portion of the polypeptide on its cell surface. In a preferred embodiment, the RNA or the DNA may be introduced using a viral expression system (e.g., vaccinia or other pox virus, retrovirus, or adenovirus), which may involve the use of a non-pathogenic (defective), replication competent virus. Techniques for incorporating RNA or DNA into such expression systems are well known to those of ordinary skill in the art. The DNA may also be “naked,” as described, for example, in Ulmer et al., Science 259:1745-1749 (1993), and reviewed by Cohen, Science 259:1691-1692 (1993). The uptake of naked DNA may be increased by coating the DNA onto biodegradable beads, which are efficiently transported into the cells.

Preferred vectors include the DNA vectors, the RNA vectors, the viral vectors such as retroviruses, lentiviruses, adenoviruses, adeno-associated viruses, poxviruses such as vaccinia virus and attenuated poxviruses such as Ankara (MVA), NYVAC, ALVAC, TROVAC, other viral vectors such as sindbis virus, cytomegalovirus and herpes simplex virus, and the bacterial vectors.

The term “expression” is used according to the invention in its most general meaning and comprises the production of RNA and/or peptides or polypeptides, e.g. by transcription and/or translation. With respect to RNA, the term “expression” or “translation” relates in particular to the production of peptides or polypeptides. It also comprises partial expression of nucleic acids. Moreover, expression can be transient or stable.

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells. The first viruses used as gene vectors were DNA viruses including the papovaviruses (simian virus 40, bovine papilloma virus, and polyoma) and adenoviruses.

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb.

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the vaccines of the present invention. AAV has a broad host range for infectivity. Details concerning the generation and use of rAAV vectors are described in U.S. Pat. Nos. 5,139,941 and 4,797,368, each incorporated herein by reference.

Retroviruses have promise as gene delivery vectors in vaccines due to their ability to integrate their genes into the host genome, transferring a large amount of foreign genetic material, infecting a broad spectrum of species and cell types and of being packaged in special cell-lines. In order to construct a retroviral vector, a nucleic acid (e.g., one encoding an antigen of interest) is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Immunogenic Composition or Vaccine Administration

To kill cells, inhibit cell growth, inhibit metastasis, decrease tumor or tissue size and otherwise reverse or reduce the malignant phenotype of tumor cells, using the methods and compositions of the present invention, one would generally administrate (or made expressed) the peptides of the invention to induce T cells that are able to recognize and kill the targeted cancer cells. The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a tumor-associated peptide, polypeptide or construct encoding therefor. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease.

Delivery via syringe or catherization is preferred. Such continuous perfusion may take place for a period from about 1-2 hr, to about 2-6 hr, to about 6-12 hr, to about 12-24 hr, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

An effective amount of the pharmaceutical immunogenic or vaccine composition, generally, is defined as that amount sufficient to detectably and repeatedly to ameliorate, reduce, minimize or limit the extent of the disease or condition or symptoms thereof. More rigorous definitions may apply for vaccine compositions, including elimination, eradication or cure of disease.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. The therapeutically effective daily amount of peptide (total amount of peptide or peptides according to the invention) administered may be in the range of 0.01 mg to 10 mg, especially 0.025 mg to 5.0 mg, or in the range of 0.025 mg to 1.0 mg.

The treatments may include various “unit doses.” Unit dose is defined as containing a predetermined-quantity of the therapeutic composition. The quantity to be administered, and the particular route and formulation, are within the skill of those in the clinical arts and may vary depending on the nature of the composition, either as a peptide composition or an expressing vector composition. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of vector and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

### BRIEF DESCRIPTION OF THE DRAWINGS

FIG. 1: A. Correlation of the expression of the 19 HERV subtypes overexpressed in TNBC with immune signatures, showing a correlation with antigen presentation, cytotoxic and immunoregulatory pathway; positive correlation is indicated by each dot (light or dark grey); B. As an example, correlation of HERV-K 10 expression with the T cell signature.

FIG. 2: A. Representative plots of dextramer staining of CD8+ T cells without or with stimulation by CMV pp65 peptide (respectively, upper quadrants) and without or with HERV peptide P5 (SEQ ID NO: 5) (respectively, lower quadrants). B. Percentage of dextramer positive CD8+ T cells at day 12 without or with stimulation with specific HERV peptides (P1 to P7—SEQ ID NO: 1 to 7) or controls on several donors' PBMCs.

FIG. 3: A. Representative plots of IFN-γ production by CD8+ T cells after contact with T2 cells pulsed with negative and positive controls (upper quadrants) and HERV peptides P1, P2, P3 (SEQ ID NO: 1, 2 and 3) (lower quadrants) B. Percentage of IFN-γ positive CD8+ T cells at day 12 without or with stimulation with specific HERV peptides (P1, P2 and P3 (SEQ ID NO: 1, 2 and 3) or controls on several donors' PBMCs.

FIG. 4: A. Representative plots of dextramer staining of CD8+ T cells without or with stimulation by CMV pp65 peptide (respectively, upper quadrants) and without or with HERV peptide P1 (SEQ ID NO: 1) (respectively, lower quadrants).

B. Fold change ratio between percentage of dextramer positive specific CD8+ T cells in the peptide stimulated condition versus non stimulated (P1 to P7—SEQ ID NO: 1 to 7) at day 12 on several donors' PBMCs.

C. Representative histograms of the number of IFN-γ+ and Granzyme-I3+ spots after 12 days stimulation and following 24h of contact with T2 cells pulsed with the cognate peptide.

FIG. 5: A. Representative plots of IFN-γ production by CD8+ T cells after contact with T2 cells pulsed with negative and positive controls (upper quadrants) and HERV peptides P1, P2, P3 (SEQ ID NO: 1 to 3) (lower quadrants), on donor a;

A (continued). representative plots of IFN-γ production by CD8+ T cells after contact with T2 cells pulsed with negative and positive controls (upper quadrants) and HERV peptides P4, P5, P6 and P7 (SEQ ID NO: 4 to 7) (lower quadrants) for donor b;

B. Percentage of IFN-γ positive CD8+ T cells at day 12 without or with stimulation with specific HERV peptides (P1 to P7—SEQ ID NO: 1 to 7) or controls on several donors' PBMCs.

FIG. 6: A. Representative plots of dextramer staining for P1 (SEQ ID NO: 1) after sorting and expansion of specific P1 CD8+ T cells (right quadrant) and their negative counterpart (left quadrant).

B. Representative histograms of the number of IFN-γ+ and Granzyme-I3+ spots, after 24h of culture of the P1 (SEQ ID NO: 1) specific CD8+ T cells with T2 cells pulsed with P1 or negative control (without peptide charged).

C. Representative curves of real-time cell death quantification in co-cultures of MDA-MB-231 cell line (pulsed or not with the peptide of interest), with P1 (SEQ ID NO: 1) specific CD8+ T cells or their negative counterpart.

D. Representative histograms of the percentage of intracellular staining of IFN-γ (PE) of P1 (SEQ ID NO: 1) specific CD8+ T cells (in black) versus their negative counterpart (non specific CD8+ T cells, in white) after 6h of co-culture with MDA-MB-231 cell line pulsed or not with the peptide of interest. The addition of an HLA-A2 blocking antibody was used as a control.

FIG. 7: A. Representative schema of dextramer positive tumor infiltrated lymphocytes (TILs) (Black quadrants) found in CD8 T cells expanded from TNBC dilacerate. P1 to P7 (SEQ ID NO: 1 to 7) represent dextramer for peptides 1 to 7, TNBC 1 to 4 represent 4 different patients B. Representative schema of dextramer positive TILs (Black quadrants) found in CD8 T cells expanded from ovarian tumor dilacerate. P1 to P7 (SEQ ID NO: 1 to 7) represent dextramer for peptides 1 to 7, Ovary 1 to 3 represent 3 different patients.

The invention will now be described using non-limiting examples, referring to the drawings.

### Example

Identification of HERV Sequences

HERVs DNA sequences from different families (including HERV-K, HERV-H, HERV-W, HERV-E and ERV3) were extracted from the Genbank database. BLAST was used to localize these sequences on the human genome (GRCH37), keeping the position with at least 98% of similarity on at least 85% of the queried sequences and no gap. 66 functional HERVs sequences have thus been identified.

Analysis of HERV Sequences in TNBC

HERVs expression was analyzed in a pre-existing database of 84 breast cancer samples containing 42 TNBC. Comparison was made with 56 normal beast sample (51 peritumoral and 5 mammal reduction samples). RNA was extracted from fresh tumor biopsies performing DNAse treatment and poly A selection. If presenting a sufficient quality (RNA integrity number>6.5), functional HERVs sequences are aligning with the RNAseq data.

Multiple component analysis of the 66 Human endogenous retroviruses (HERV) subtypes was performed. 42 HERVs are expressed. 19 HERVs specifically characterize triple negative breast cancer (TNBC) upon normal tissue and ER+ subtypes: HERVK 10, 17, 22, 7, 6, 21, 25, 11, 20, 16, 23, 1, 5; HERVH_4, 7; and HERV3_1.

Peptide Selection and Synthesis

Common regions in the Gag and Pol shared between the 19 overexpressed HERVs were identified after alignement of the reads on a reference genome. Using different epitope prediction tools (NetMHC I & II), potential strong epitope binders for the most frequent MHC I and II alleles were identified. Among them, 7 predicted 9-mer strong binders for HLA-A*0201 were selected and synthetized: 4 Gag and 3 Pol Peptides (JPT peptide technology, Berlin, Germany). Peptides identity was confirmed by mass spectrometry by the seller. Purity>95% was expected and determined by high-performance liquid chromatography. Lyophilised peptides were dissolved in deionized water <5% DMSO, aliquoted and conserved at −20° C. until use.

29mer GMX peptides (SEQ ID NO: 8-14) containing the 9-mer peptides strong binder for class I MHC (SEQ ID NO: 1-7), plus lateral sequences of class II motifs (10-mer on each side, except for peptide SEQ ID NO: 12, where the sequence SEQ ID NO: 5 is on the C-terminal) were identified and analyzed for synthesis.

Bioinformatics Analysis of the Correlations Between HERVs Expression and T Cell Signatures

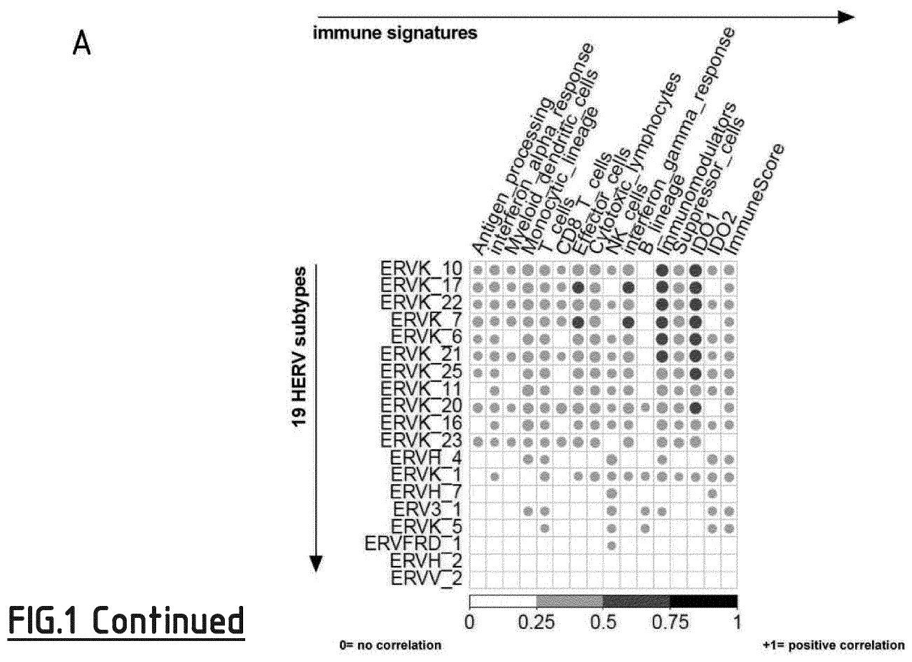

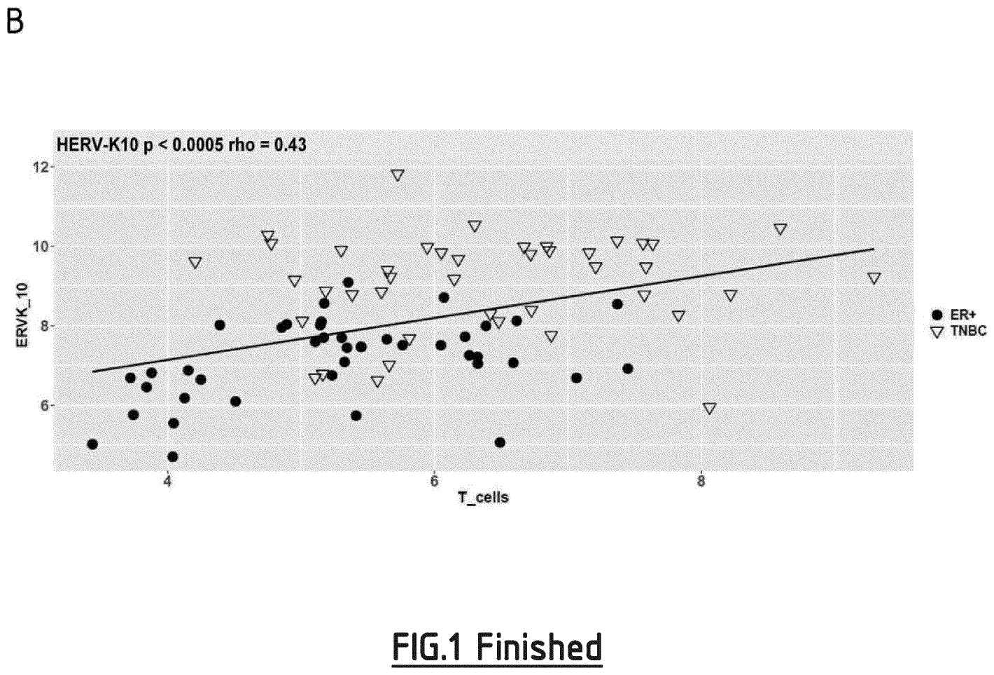

Different signatures were used to evaluate the immune characteristics of the tumor: MCP counter signatures (ref http://cit.ligue-cancernet/?p=1338&lang=en) for T cells, CD8 T cells, NK cells, Cytotoxic lymphocytes, as well as Fibroblasts, Neutrophils, and Endothelial cells for a control analysis; Estimate package (http://bioinformatics.mdanderson.org/estimate/index.html) for ImmuneScore, StromalScore; Immunophenogram 15 for Effector cells, immunomodulators (immune checkpoints), suppressor cells (regulatory T cells and MDSCs). Specific genes will be also evaluated (like OCT4, TRIM28, SETDB1) as well as EMT signature (SSGSEA and Jean-Paul Thierry signature) and signatures associated specifically with the tumor subtype. Correlation between these signatures and HERVs will be analyzed using classical statistical methods. FIG. 1A shows a significant correlation between 19 HERVs and specific immune signatures as antigen presentation-related, cytotoxic or immunomodulatory signatures. As an example, HERV-K 10 expression strongly correlates with the T cell signature in breast cancer (FIG. 1B).

PBMCs Culture for Specific CD8+ HERV+ Stimulation

PBMCs from HLA-A2 donors where cultured for 12 days in AIM-V medium (Thermo Fisher Scientific) supplemented with 5% AB human serum (pool of 5 donors from EFS Lyon, filtered) and 20 UI/mL IL-2 (PROLEUKIN aldesleukine, Prometheus, Vevey, Switzerland) enriched with 5 μg/mL of R848 (InvivoGen, San Diego, USA) and 10 μg/mL of Poly-IC (InvivoGen) and the peptide of interest (10 μM). Cultures were performed in a 96 U-bottom wells plate, 1.5×10⁵ cells per well, and 20 wells were performed for each peptide condition. 100 μL of medium were changed and enriched with R848, Poly-IC, IL-2 and the peptide of interest (peptides of sequences SEQ ID NO: 1-7) to achieve the same final concentration on day 3. IL-2 and the peptide of interest where added on day 6 and on day 10 IL-2 only. Positive control was cultured with 0.1 μg/mL of PP65 (JPT peptide technology) in the dextramer experiment, a CMV peptide that is presented by the MHC class I and specifically stimulate CD8+ T cells. For IFN-γ experiment, 0.4 μg/mL CEF peptide (Mabtech) were used, consisting in a pool of 23 MHC class I restricted viral peptides from human CMV, EBV and influenza virus which stimulate CD8+ T cells to preferentially synthetize IFN-γ.

Dextramer Assay and Sorting

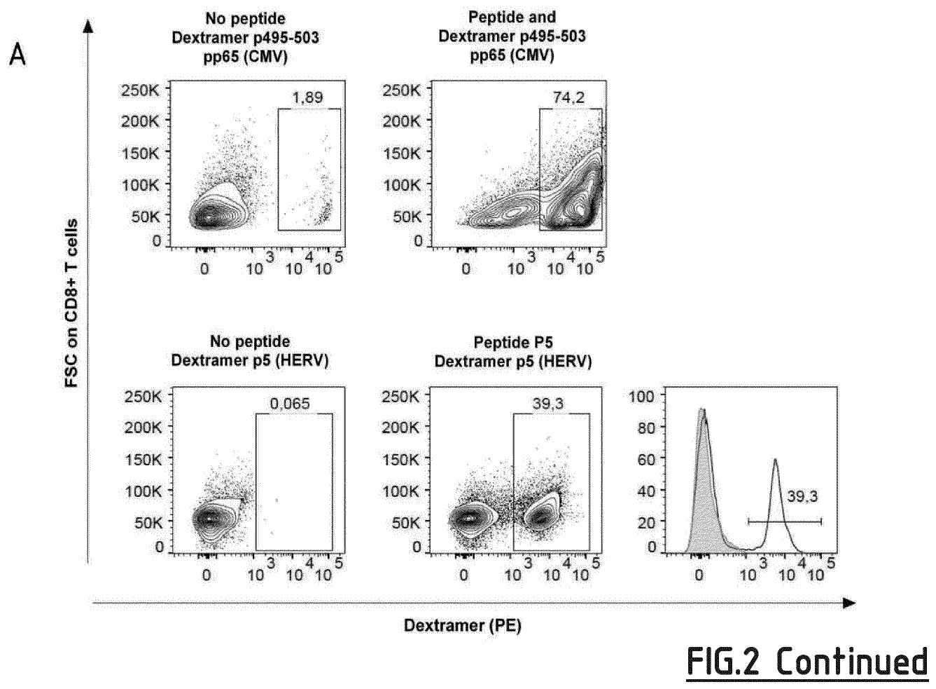

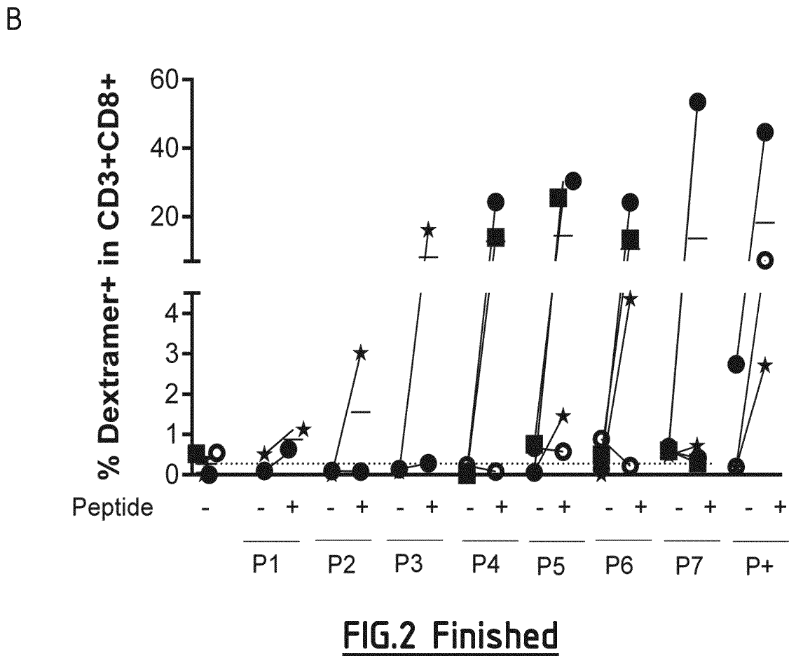

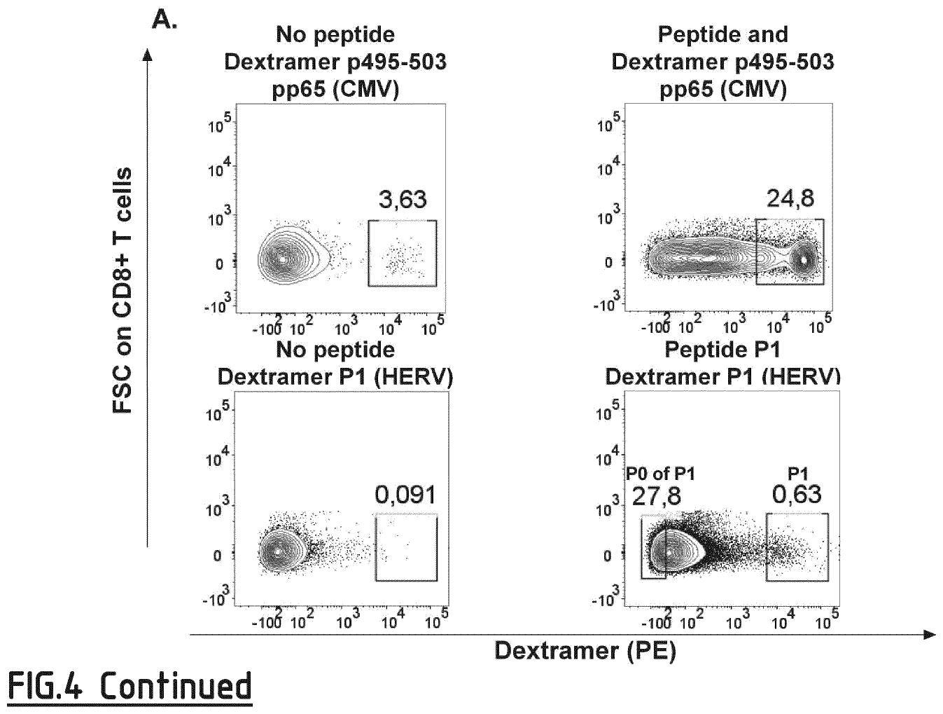

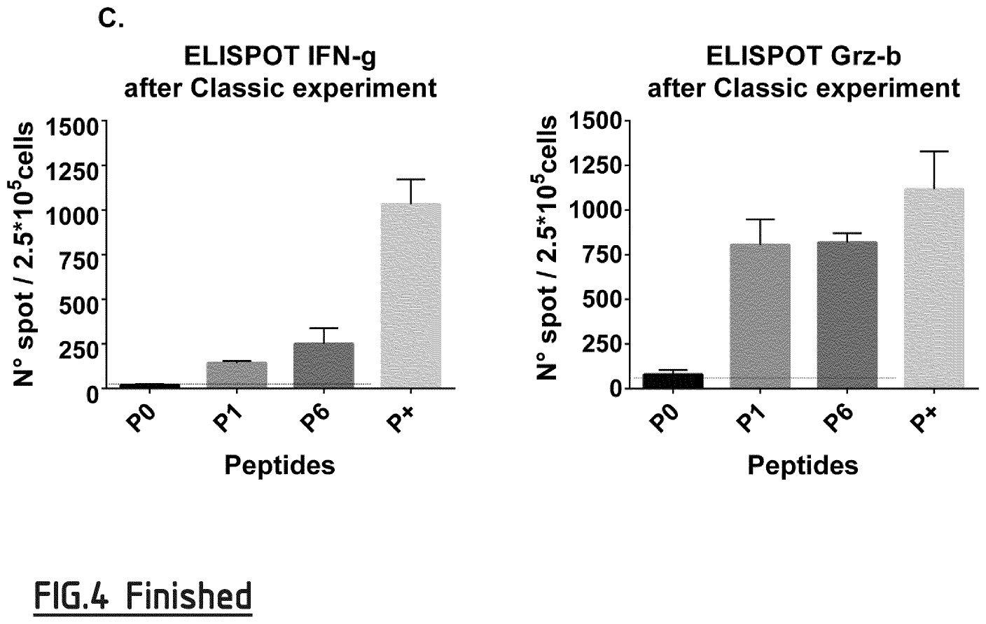

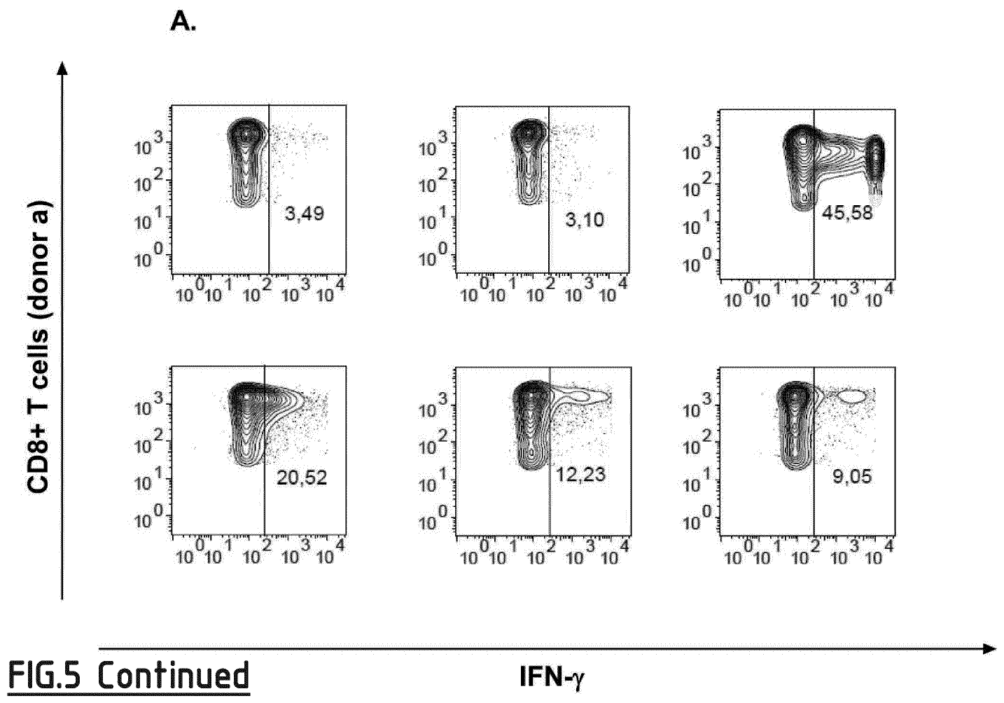

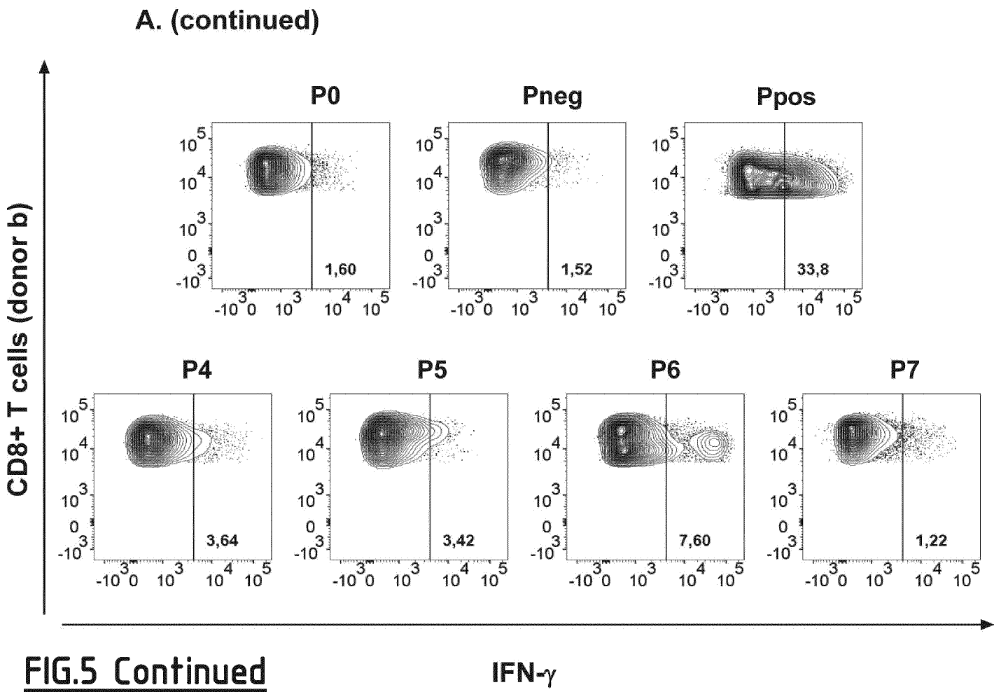

On day 12, cells from same conditions were pooled together in polypropylene tubes, washed with 2 mL of FACS buffer and resuspended in FACS buffer. Conditions were stained with 10 μL of the corresponding dextramer (Immudex, Copenhague, Danemark) for 15 minutes at room temperature in the dark. Zombie Near Infra-Red (NIR) fixable viability kit (Zombie NIR, biolegend, Paris, France) was used at 1/400 to assess viability. Anti CD3 (BV421, Biolegend) and anti CD8 (FITC, Beckman coulter, Brea, USA) antibodies were then added to each condition ( 1/10 in the assay of FIG. 2, 1/25 for FIG. 4) and left 20 minutes in the dark at 4° C. Cells were then washed two times with 2 ml of FACS buffer and resuspended in 350 μL of FACS buffer until analysis. Analysis was performed on FACS Fortessa (BD) to discriminate multimer specific HERV CD8+ T cells.

Results in FIG. 2A show the population stained by dextramer in non-specific (left panels) and specific (right panels) peptide-pulsed PBMCs. In upper panels, up to 72% of CD8+ T cells resulted positive after stimulation with the positive control pp65 peptide from CMV vs. 2.02% in non-stimulated PBMCs (indicating a possible presence of memory T cells against CMV due to previous infections). Interestingly PBMCs stimulated with a HERV peptide (e.g. SEQ ID NO: 5 in the lower panel) resulted in a 34.2% of dextramer positive CD8+ T cells vs. 0.04% in the non-stimulated condition. Results obtained on four different donors are summarized in FIG. 2B.

Results in FIG. 4A show the population stained by dextramer in non-specific (left panels) and specific (right panels) peptide-pulsed PBMCs. In upper panels, up to 23.40% of CD8+ T cells resulted positive after stimulation with the positive control pp65 peptide from CMV vs. 3.63% in non-stimulated PBMCs (indicating a possible presence of memory T cells against CMV due to previous infections). Interestingly PBMCs stimulated with a HERV peptide (e.g. SEQ ID NO: 1 in the lower panel) resulted in a 0.63% of dextramer positive CD8+ T cells vs. 0.091% in the non-stimulated condition. Results obtained on 12 different donors are summarized in FIG. 4B, showing a significant increase in dextramer positive CD8+ T cells for P1, P4 and P6 (e.g. SEQ ID NO: 1, 4, 6) and slightly for P2 and P3 (e.g. SEQ ID NO: 2, 3)

Peptide stimulated PBMCs after the 12-day culture were used to perform an ELISPOT assay for IFN-γ and Granzyme-β (FIG. 2C). 2.10⁵ cells per well were put in co-culture with T2 cell specific for the peptide of interest in a ratio 10:1. ELISPOT was revealed after 24h showing specific IFN-γ and Granzyme-β spots for P1 and P6 (e.g. SEQ ID NO: 1, 6).

Cytotoxicity Assay with T2 Cells Contact

On day 12 of PBMCs culture, T2 cells were washed in RPMI and resuspended into AIM-V medium (Thermo Fisher Scientific). T2 (SD Cell line) are a lymphoblast cell line deficient in the transporter associated with antigen processing (TAP) protein, and therefore cannot present endogenous peptides on the class I MHC, but can be used to monitor the CTL response to an exogenous antigen of interest in a non-competitive environment. T2 cells were first pulsed with HERV peptide by adding 10 μg/mL of corresponding peptide to 2M T2 cell for 2 hours at 37° C. PBMCs were pooled and counted and put in co-culture with corresponding T2 cells at a respective concentration of 1:5 into a new 96 U-well plate.

After 4 hours of incubation at 37° C., cells from co-culture were washed and pooled in V-well plate according to their staining condition in FACS buffer. Zombie NIR (Biolegend) was used at 1/400 to assess viability. Anti CD3 (PercP, Biolegend) and anti CD8 (FITC, Beckman coulter) antibodies were added 1/10 per condition and left for 25 minutes at 4° C. Cells were washed again and then fixed with fixation/permeabilization solution kit (Invitrogen, Carlasbad, USA) according to the manufacturer's instructions for 15 minutes at room temperature. Cells were washed two times in FACS buffer and kept at 4° c.

On day 13, cells were permeabilized with the permeabilization solution kit (Invitrogen) for 5 minutes at room temperature and anti IFN-γ (PE, Biolegend) antibody was added 1/20 to the solution for additional 25 minutes at 4° C. Cells were washed two times and resuspended in 350 μL of FACS buffer before FACS analysis. Analysis was performed on FACS Fortessa (BD) to explore the specific cytotoxicity and degranulation against T2 cells expressing HERV sequences.

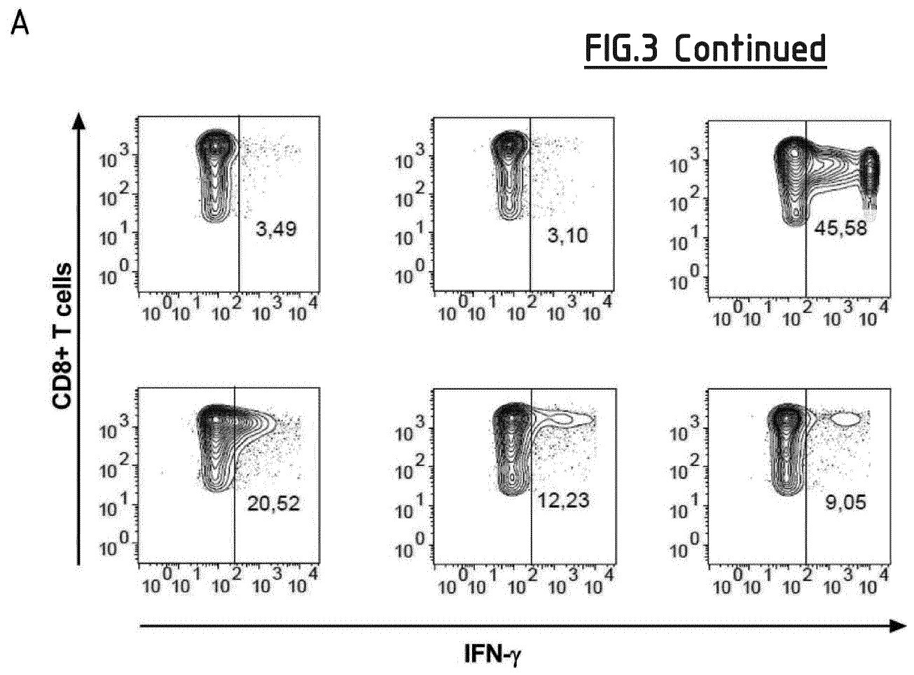

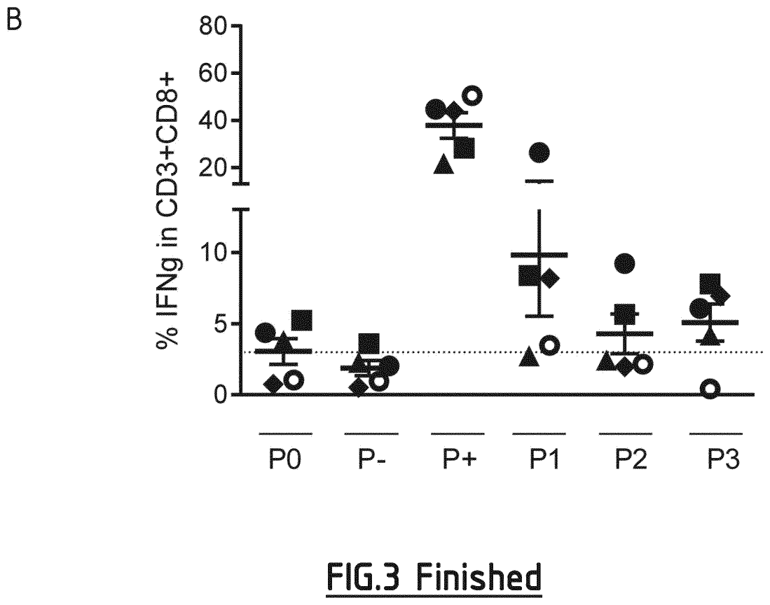

Results in FIG. 3A show IFN-γ production of stimulated CD8+ T cells against T2 cells not pulsed (P0: upper-left panel), pulsed with a different peptide (Pneg: upper-central panel), or with each cognate peptide (positive control Ppos: upper/right panel, HERV peptides of SEQ ID NO 1, 2 and 3 in lower panels); results show a specific IFN-γ production against T2 cells expressing HERV peptides. Results of 5 different donors are summarized in FIG. 3B, showing an IFN-γ production in ˜10% (in median) of CD8+ T cell stimulated with P1 (SEQ ID NO: 1) and ˜5% (in median) of CD8+ T cells stimulated with P2 or P3 (SEQ ID NO: 2 and 3).

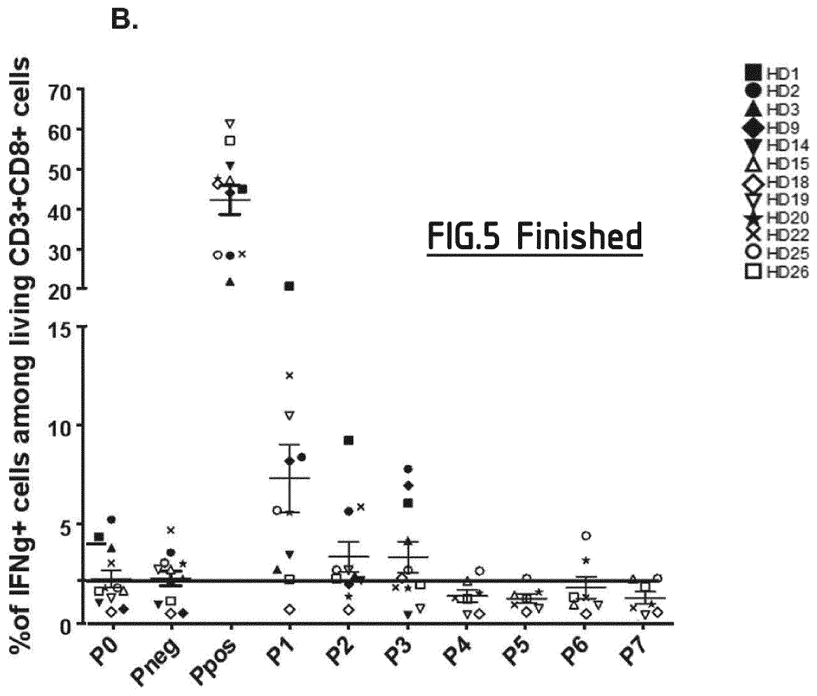

Results in FIG. 5A show IFN-γ production of stimulated CD8+ T cells against T2 cells not pulsed (P0: upper-left panel in both donor a and donor b quadrants), pulsed with a different peptide (Pneg: upper-central panel in both donor a and donor b quadrants), or with each cognate peptide (positive control Ppos: upper/right panel, HERV peptides of SEQ ID NO 1 to 3 in lower panels for donor a and HERV peptides of SEQ ID NO 4 to 7 in lower panels for donor b); results show a specific IFN-γ production against T2 cells expressing HERV peptides. Results of 12 different donors are summarized in FIG. 5B, showing an IFN-γ production in a significant number of CD8+ T cells stimulated with P1 (SEQ ID NO: 1), P2 and P3 (SEQ ID NO: 2 and 3) and in a moderate number of CD8+ T cells stimulated with P6 (SEQ ID NO: 6).

Cytotoxicity of P1 Specific CD8+ T Cells

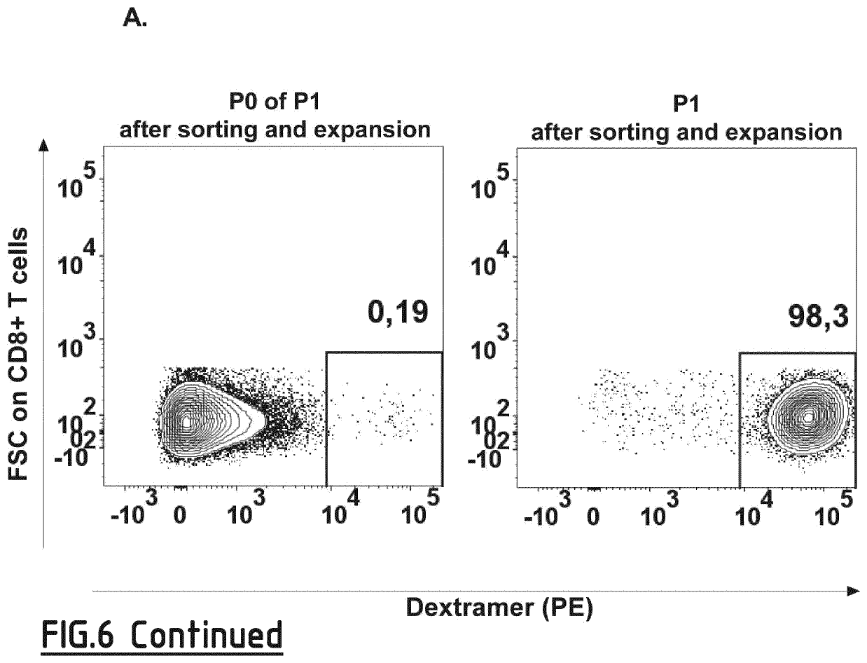

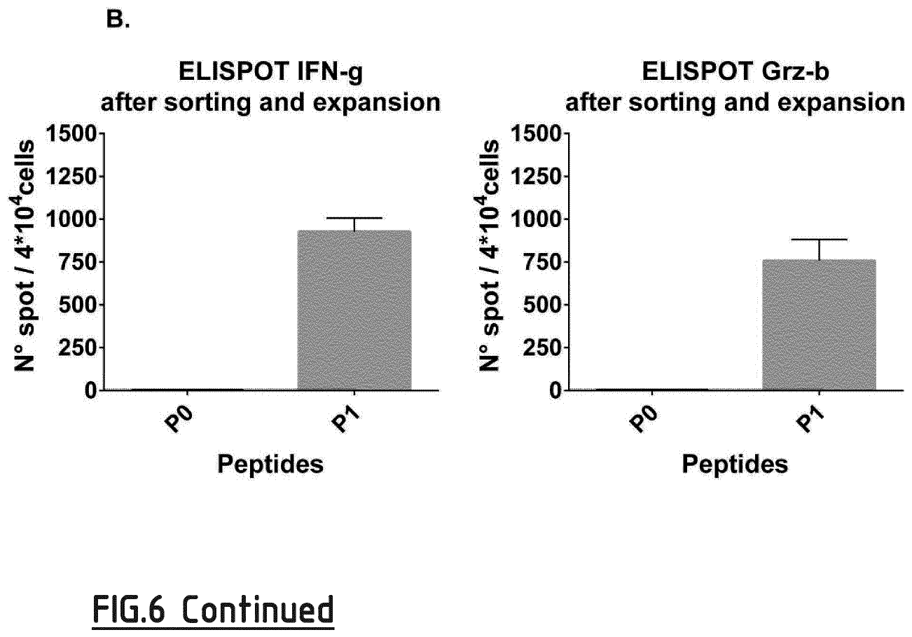

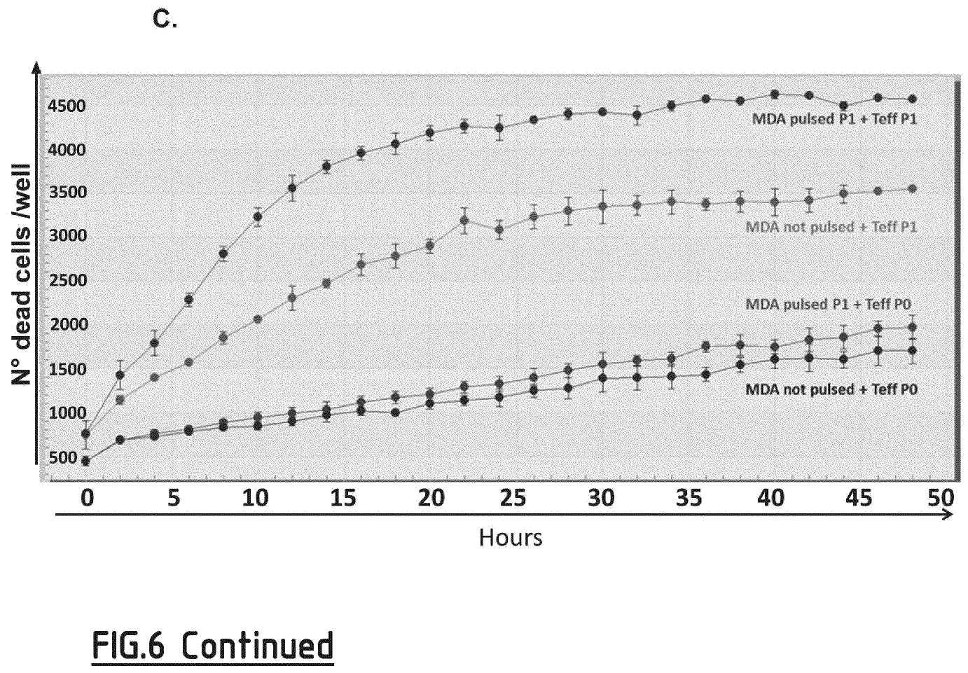

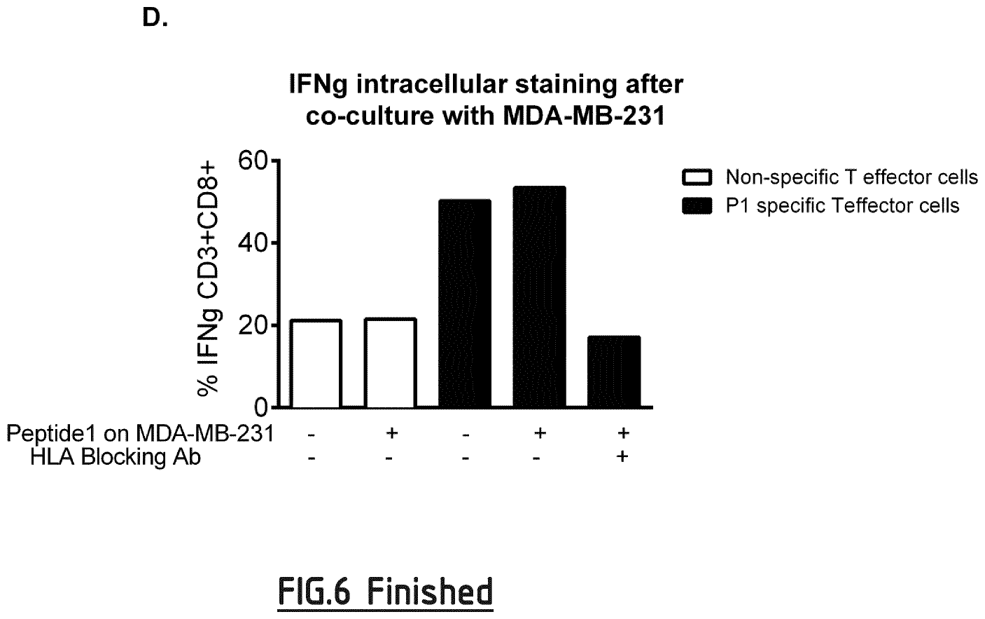

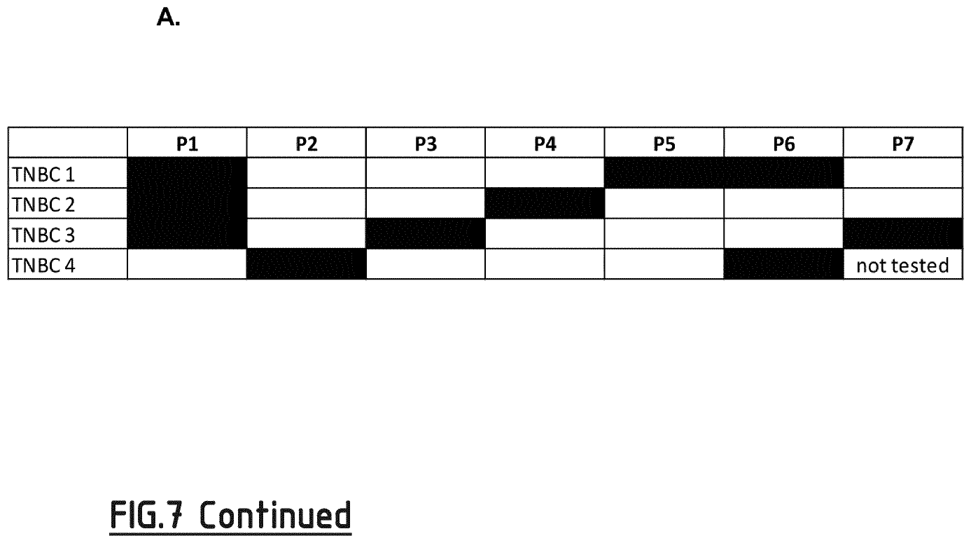

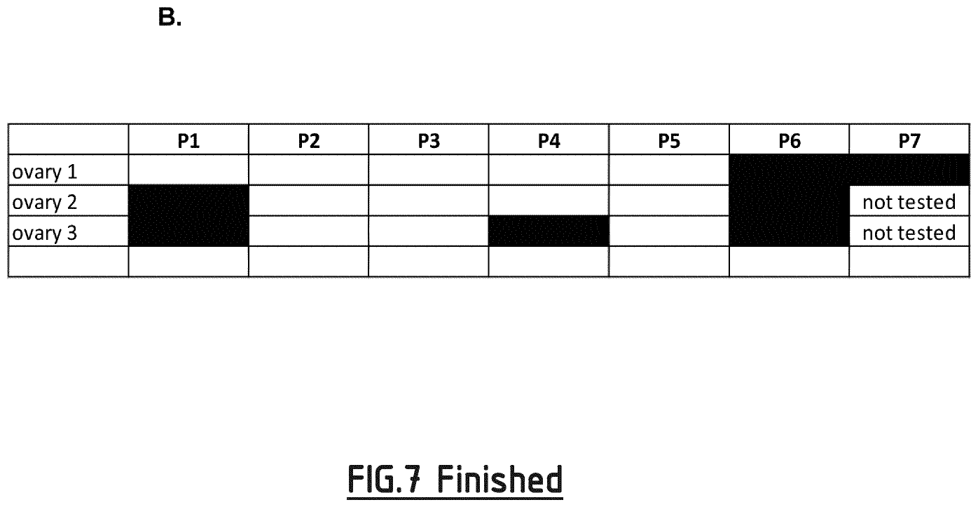

The dextramer-stained cells were sorted by FACS Aria (BD) to separate peptide-specific CD8+ T cells from the unspecific counterpart. Both fractions were collected and expanded separately on feeder cells in 96 round-well plates for 14 days. The purity of the specific versus unspecific fraction was evaluated at day 14 (FIG. 6A) resulting in >90% of dextramer positive CD8+ T cells in the positive fraction versus <5% in the negative fraction. These cells were used for the cytotoxicity experiments.

Sorted and expanded CD8+ T cells were evaluated for their cytotoxic potential by ELISPOT assay (Cellular technology limited, CTL). 4.10⁴ P1 specific CD8+ T cells were co-cultured with T2 cells previously pulsed with the peptide P1. The number of spots counted indicates a production of IFN-γ and Granzyme-β (˜800 spots for both cytokines) by the P1 specific CD8+ T cells against target cells pulsed with the cognate peptide in comparison to negative control (FIG. 6B). These experiments indicate that those cells are specific and functional.

The in silico analysis of HERV expression performed on the cell lines HMEC (HLA-A2 human mammary epithelial cells) and MDA-MB-231 (HLA-A2 Triple negative breast cancer cell line), showed an overexpression of HERVs in the MDA-MB-231 cell line in comparison to the HMEC.

The HLA-A2 TNBC cell line MDA-MB-231 was used as target for a real-time analysis of cell death induced by P1 specific CD8+ T cells. 5.10³ MDA-MB-231 cells were pulsed or not with the peptide P1 and were allowed to adhesion in 96-well plates. After adhesion P1 CD8+ T cells or their negative counterpart (control) were added into the wells. The co-culture was performed in the presence of the Cytotox green dye (Essenbioscience) which enters into the cells when the plasma membrane integrity diminishes, yielding a 100-1000-fold increase in fluorescence upon binding to deoxyribonucleic acid (DNA). The kinetics shows a very significant increase in cell death when MDA-MB-231 are co-cultured with the P1-specific T cells in comparison to their negative counterpart. As expected, a further increase of cell death was observed when target MDA-MB-231 cells were pulsed with P1 and co-cultured with P1-specific T cells (probably due to an increase in the number of HLA-peptide 1 complexes on target cells) (FIG. 6C).

Furthermore, after 6 hours of co-culture between P1 specific CD8+ T cells or their negative counterpart and MDA-MB-231 HLA-A2 TNBC cell line, an intracellular staining of IFN-γ was performed by FACS (FIG. 6D). Results shows an increase in the percentage of IFN-γ producing cells when the P1 specific cells are in the co-culture in comparison to the co-cultures with the negative counterpart. This percentage is slightly increased when MDA-MB-231 are previously pulsed with P1. The use of an anti HLA-A2 specific blocking antibody can reverse this effect, demonstrating its specificity.

Altogether, these experiments show that P1-specific CD8+ T cells specifically recognize and are functional against target cells presenting the cognate peptide (T2 cells in this experiment) and specifically recognize and kill tumor cells expressing endogeneously HERV-derived antigens (MDA-MB-231 TNBC cell line in this experiment).

Dextramer Staining of Tumor Infiltrated Lymphocytes (TILs) from TNBC and Ovarian Cancer

Tumors were dilacerated in small pieces and digested with collagenase IV and DNAse for 45 minutes. The cells obtained were resuspended in 5% human serum RPMI and distributed in 96 well (5.10⁴ cells per well). Anti-CD3/CD28 microbeads (Miltenyi biotech) were added to the well in a ratio of 1:4 with cells in the presence of IL-2 (F.C. 100 IU/ml). TILs were cultured for 14 days by changing medium at day 5, 7, 9 and 12 and the number of the cells was adapted to have 0.5×10⁶ cells/ml.

At day 14 a dextramer staining was performed (see dextramer assay paragraph) for the 7 peptides of interest (P1 to P7—SEQ ID NO: 1 to 7) and dextramer specific CD8+ T cells were identified by FACS analysis in TNBC (FIG. 7A) and ovarian cancer (FIG. 7B). Results show that specific CD8+ T cells for all the peptides of interest can be found in tumors, with some variations according to the sample. This confirms the in vivo immunogenicity of the peptides and shows that these peptides can be naturally processed and can elicit a detectable immune response during tumor development. CD8+ T cells specific for P1 and P6 are more frequently represented in the tumors and are peptides of choice as a basis of a vaccine or immunogenic composition. These results also militates in favor of a combination of several peptides in order to provide for a vaccine or an immunogenic composition that is usable in patients of unknown status with respect to this reactivity, and preferably comprising P1 and/or P6.

1. A composition comprising
2, 3, 4, 5, 6 or 7 peptides, or one or more expression vector(s) that induce(s) expression of said 2, 3, 4, 5, 6 or 7 peptides in vivo, the peptides having from 9 to 100 amino acid residues, each one comprising at least one of the epitopes of sequences SEQ ID NO: 1-7, and each peptide comprising at least one different epitope with respect to the others; or
at least one peptide, or an expression vector that induces expression of said at least one peptide in vivo, said peptide having from 9 to 100 amino acids residues and comprising 2, 3, 4, 5, 6 or 7 of the epitopes of sequences SEQ ID NO: 1-7;
and a pharmaceutically acceptable vehicle or excipient.

2. The composition of claim 1, comprising or expressing one or more peptides comprising the epitopes of sequence SEQ ID NO: 1 and/or 6.

3. The composition of claim 1, comprising or expressing 3, 4, 5, 6, or the 7 peptides comprising the epitopes of sequences SEQ ID NO: 1 to 7.

4. The composition of claim 1 wherein the peptides comprise 9 to 100, 70, 50, 40, 30, 25, or 20 amino acid residues and at least one of said epitopes of sequence SEQ ID NO: 1 to 7.

5. The composition of claim 4, wherein each peptide of 9 to 100, 70, 50, 40, 30, 25, or 20 amino acid residues comprises one specific epitope of SEQ ID NO: 1, 2, 3, 4, 5, 6 or 7.

6. The composition of claim 1, wherein the peptide(s) comprise(s) 9 to 100, 70, 50, 40, 30, 25, or 20 consecutive amino acid residues of an HERV gag or pol including at least one of said epitopes of sequence SEQ ID NO: 1 to 7.

7. The composition of claim 1, wherein the peptide(s) comprise(s) 13, 14, 15, 16, 17, or 18, consecutive amino acid residues of an HERV gag or pol including at least one of said epitopes of sequence SEQ ID NO: 1 to 7.

8. The composition of claim 1, wherein the peptides are selected from the group consisting of SEQ ID NO: 8-14 and the composition comprises 2, 3, 4, 5, 6 or 7 of them.

9. The composition of claim 1, wherein the peptides consist of 9, 10, 11, 12, 13, 14, 15, 16, 17, 18, 19, 20, 21, 22, 23, 24, 25, 26, 27, 28 consecutive amino acid residues of sequences SEQ ID NO: 8-14, including the 9-mer epitope, and the composition comprises 2, 3, 4, 5, 6 or 7 of them.

10. Vaccine or immunogenic composition comprising the composition of claim 1 and a pharmaceutically acceptable vehicle or excipient and preferably an adjuvant.

11. Composition comprising Cytotoxic T Lymphocytes (CTLs) of a patient treated with a peptide as described in claim 1, or comprising T-cell Receptor (TCR) engineered T cells recognizing a peptide as described in any one of claims 1-7, and a pharmaceutical vehicle.

12. Composition according to claim 1, for use in treating cancer, in particular breast cancer, including triple negative breast cancer, ovarian cancer, melanoma, sarcoma, teratocarcinoma, bladder cancer, lung cancer (non small cell lung carcinoma and small cell lung carcinoma), head and neck cancer, colo-rectal cancer, glioblastoma and leukemias.

13. Isolated peptide selected from the group consisting of the peptides of sequence SEQ ID NO: 2-14, and the peptides having 10 to 100, 70, 50, 40, 30, 25, or 20 amino acids and comprising at least one of said peptides of SEQ ID NO: 2-7.
